(19) United States
(12) Patent Application Publication
(10) Pub. No.: US 2018/0154155 A1
(43) Pub. Date: Jun. 7, 2018

(54) NEUROMODULATION DEVICES FOR DELIVERING NEUROMODULATION ENERGY TO PROXIMAL VASCULAR PORTIONS AND DISTAL VASCULAR PORTIONS AND ASSOCIATED SYSTEMS AND METHODS

(71) Applicant: Medtronic Ardian Luxembourg S.a.r.l., Luxembourg (LU)

(72) Inventors: James Keaveney, Galway (IE); Ultan Horgan, Galway (IE); Jeffrey Madden, Galway (IE)

(21) Appl. No.: 15/368,895

(22) Filed: Dec. 5, 2016

(51) Int. Cl. A61N 1/36; A61N 1/05; A61N 5/04; A61N 5/06; A61F 7/12; A61N 7/02; A61B 18/18; A61B 18/14

(52) U.S. Cl. CPC A61N 1/36117; A61B 2018/00577; A61N 1/0551; A61N 5/045; A61N 5/0603; A61N 5/0622; A61F 7/12; A61N 7/02; A61B 18/1815; A61B 18/1492; A61N 2005/0612; A61N 2005/0659; A61N 2005/0662; A61F 2007/126; A61B 2018/1861; A61B 2018/1807; A61N 1/36007

(57) ABSTRACT

Neuromodulation devices for delivering neuromodulation therapy to distal vascular portions and proximal vascular portions and associated systems and methods. A neuromodulation device can include an elongated shaft having a proximal portion and a distal portion, each portion being sized and shaped for simultaneous positioning and deployment within respective proximal and distal vascular portions of a human vasculature. The elongated shaft can have an outer cross-sectional dimension that decrementally changes towards the distal end. The elongated shaft can also have a plurality of sections, each section configured to be independently shaped upon deployment in the intended distal or proximal vascular portion.

### TECHNICAL FIELD

The present technology is related to neuromodulation devices. In particular, at least various embodiments of the present technology are related to neuromodulation devices for delivering neuromodulation energy to proximal vascular portions and distal vascular portions.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS in particular has been identified experimentally and in humans as a likely contributor to the complex pathophysiology of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is an exploded view of a neuromodulation device configured in accordance with an embodiment of the present technology.

FIG. 2 is a side view of the distal portion of the neuromodulation device of FIG. 1 in an expanded state in accordance with an embodiment of the present technology.

FIG. 3 is a side view of the distal portion of a neuromodulation device of FIG. 1 positioned within a blood vessel in accordance with an embodiment of the present technology.

FIGS. 4-6 are enlarged views of braided regions of the distal portion of the neuromodulation device of FIG. 1 configured in accordance with embodiments of the present technology.

FIGS. 7-9 are enlarged cross-sectional views of the distal portion of the neuromodulation device of FIG. 1 taken along cross-sections 7-7, 8-8 and 9-9 of FIG. 1 in accordance with other embodiments of the present technology.

FIG. 10 is an isometric view of a distal portion of a neuromodulation device configured in accordance with another embodiment of the present technology.

FIG. 11 is a side view of the distal portion of the neuromodulation device of FIG. 10 positioned within a blood vessel in accordance with another embodiment of the present technology.

FIG. 12 is an isometric view of a distal portion of a neuromodulation device configured in accordance with a further embodiment of the present technology.

FIGS. 13 and 14 are enlarged transverse cross-sectional views of portions of the distal portion of the neuromodulation device of FIG. 12 configured in accordance with a further embodiment of the present technology.

FIG. 15 is a side view of the distal portion of the neuromodulation device of FIG. 12 positioned within a blood vessel in accordance with a further embodiment of the present technology.

FIG. 16 is a partially schematic illustration of a neuromodulation system configured in accordance with an embodiment of the present technology.

FIG. 17 illustrates modulating renal nerves with a neuromodulation device described herein in accordance with an additional embodiment of the present technology.

FIG. 18 is a conceptual illustration of the sympathetic nervous system (SNS) and how the brain communicates with the body via the SNS.

FIG. 19 is an enlarged anatomic view of nerves innervating a left kidney to form the renal plexus surrounding the left renal artery.

FIGS. 20 and 21 are anatomic and conceptual views, respectively, of a human body depicting neural efferent and afferent communication between the brain and kidneys.

FIGS. 22 and 23 are anatomic views of the arterial vasculature and venous vasculature, respectively, of a human.

### DETAILED DESCRIPTION

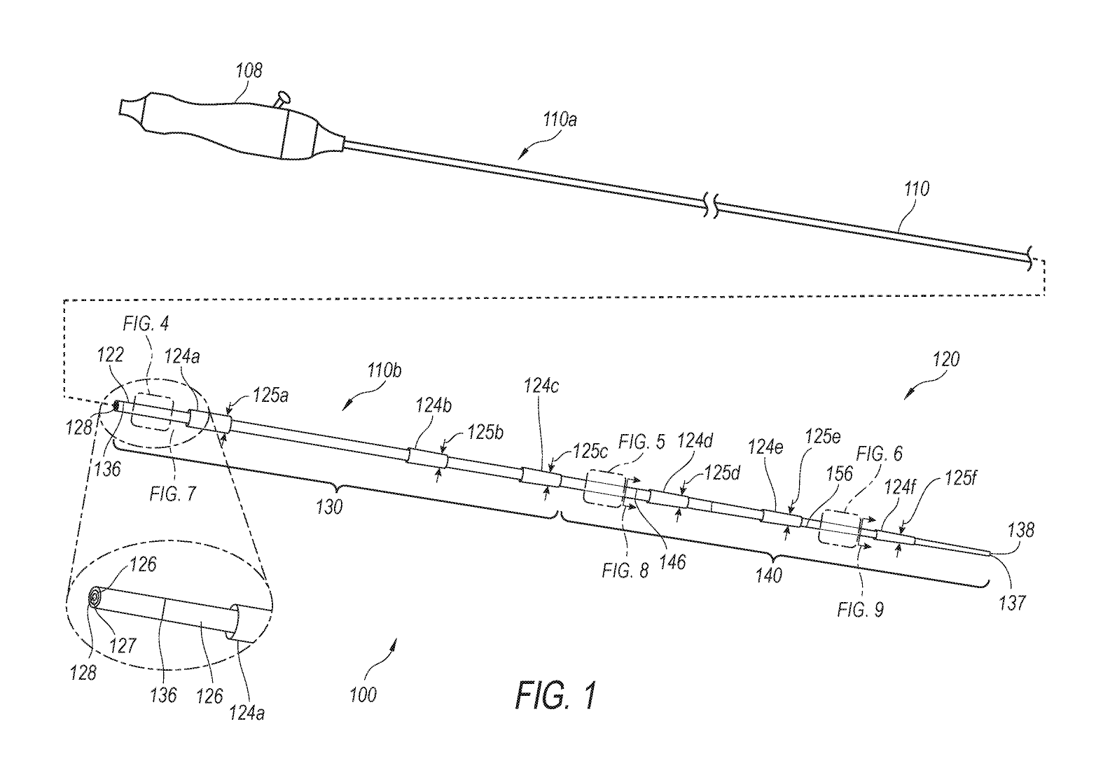

Neuromodulation devices and systems configured in accordance with several embodiments of the present technology can include a neuromodulation assembly having a variable outer cross-sectional dimension to deliver neuromodulation energy to both proximal vascular portions and distal vascular portions. In certain embodiments, an outer cross-sectional dimension of the neuromodulation assembly decrementally changes towards a distal end portion of the assembly. The neuromodulation assembly can include energy delivery elements configured to deliver neuromodulation energy to both a proximal vascular portion having a first diameter and a distal vascular portion having a second diameter less than the first diameter. Specific details of several embodiments of the present technology are described herein with reference to FIGS. 1-23. Although many of the embodiments are described with respect to devices, systems, and methods for intravascular renal neuromodulation, other applications and other embodiments in addition to those described herein are within the scope of the present technology. For example, at least some embodiments may be useful for intraluminal neuromodulation, for non-renal neuromodulation, and/or for use in therapies other than neuromodulation. In addition, embodiments of the present technology can have different configurations, components, and/or procedures than those shown or described herein. Moreover, a person of ordinary skill in the art will understand that embodiments of the present technology can have configurations, components, and/or procedures in addition to those shown or described herein, and that these and other embodiments can be without several of the configurations, components, and/or procedures shown or described herein without deviating from the present technology.

As used herein, the terms “distal” and “proximal” define a position or direction with respect to a clinician or a clinician's control device (e.g., a handle of a neuromodulation device). The terms, “distal” and “distally” refer to a position distant from or in a direction away from a clinician or a clinician's control device. The terms “proximal” and “proximally” refer to a position near or in a direction toward a clinician or a clinician's control device. The headings provided herein are for convenience only and should not be construed as limiting the subject matter disclosed.

Additionally, the term “distal vascular portion” refers to any distal portion of a vessel (e.g., renal artery) having a cross-sectional dimension less than a cross-sectional dimension of a proximal portion of the vessel, and/or a different vessel extending from the. In some embodiments, the distal vascular portion of the renal artery can refer to an auxiliary vessel, a branch of the renal artery (e.g., anterior and/or posterior), a suprarenal artery, and/or a segmental artery (e.g., superior, inferior, anterior, and/or posterior).

Further, the term “proximal vascular portion” refers to any proximal portion of a vessel (e.g., renal artery) having a cross-sectional dimension greater than a cross-sectional dimension of a distal portion of the vessel, and/or a different vessel extending from the vessel. In some embodiments, the proximal vascular portion of the renal artery can refer to the superior vena cava, left brachiocephalic vein, right brachiocephalic vein, azygos vein, azygos arch, or other vessel suitable for neuromodulation therapy.

### I. Selected Embodiments of Neuromodulation Devices and Assemblies for Proximal Vascular Portions and Distal Vascular Portions and Associated Methods

Renal neuromodulation therapy aims to modulate the autonomic nervous system, specifically the SNS, by modulating or destroying renal efferent sympathetic nerves and afferent renal sensory nerves. Ablating the renal sensory nerves at a target therapy site in a distal vascular portion, e.g., vessels branching from the renal artery, has been shown to increase the efficacy of renal neuromodulation therapy. However, delivering renal neuromodulation therapy to the target therapy site in the distal vascular portion can be challenging for at least two reasons. First, the target therapy site can be difficult to access when the cross-sectional dimension of the distal vascular portion vessel is too small to accommodate existing neuromodulation therapy devices. Second, the amount of energy delivered by electrodes of existing neuromodulation therapy devices can damage distal vascular portions and/or surrounding tissue. Accordingly, beneficial therapy site(s) in distal vascular portions may remain untreated. Therefore, the efficacy of many procedures can be improved by renal neuromodulation devices sized and shaped to deliver neuromodulation therapy to treatment sites in both a proximal vascular portion and a distal vascular portion. The present technology includes several embodiments of neuromodulation devices, in particular, neuromodulation assemblies, sized and shaped for delivery into both proximal vascular portion and a distal vascular portion. These neuromodulation devices are configured to deliver desired amounts of neuromodulation energy to therapy locations in the proximal vascular portion and a distal vascular portion in accordance with the present technology.

FIG. 1 is an exploded view of a neuromodulation device 100 in accordance with an embodiment of the present technology, including a partially schematic enlarged isometric view of a distal portion of the device. The neuromodulation device 100 includes a handle 108, an elongated shaft 110 having a proximal portion 110a and a distal portion 110b, and a neuromodulation assembly 120 at the distal portion 110b of the elongated shaft 110. The elongated shaft 110 and the neuromodulation assembly 120 can be 2, 3, 4, 5, 6, or 7 French size or one or more other suitable catheter sizes. The neuromodulation assembly 120 includes a plurality of energy delivery elements 124 mounted on a support member 122 that can be shaped into a desired configuration in operation. The support member 122 is configured to position the energy delivery elements 124 in both a proximal vascular portion and a distal vascular portion at the same time.

In the embodiment illustrated in FIG. 1, the neuromodulation assembly 120 includes at least a first section 130 and a second section 140 distal to the first section 130 with both sections extending along a length of the support member 122. In the embodiment shown in FIG. 1, the energy delivery elements 124 are present in both the first section 130 and the second section 140. In other embodiments, the support member 122 can include more sections than the first section 130 and the second section 140.

The dimensions (e.g., length and outer cross-sectional dimensions) of the support member 122 in the embodiments described herein are selected to accommodate vascular structures or other body lumens with different proximal and distal shapes and sizes. The length of the support member 122 may be selected such that the expanded support member 122 is longer than a proximal vascular portion (e.g., a main renal artery, which is typically greater than 7 cm) and extends into a distal vascular portion (e.g., a smaller downstream portion of the main renal artery or the renal branches) extending from the proximal vascular portion. In accordance with several embodiments of the present technology, the support member 122 has an outer cross-sectional dimension that decreases along the length towards the distal end 137 such that the neuromodulation assembly 120 can be positioned and deployed in the proximal vascular portion and the distal vascular portion in a single deployment. In these embodiments, the outer cross-sectional dimension of the support member 122 can be different at the first section 130 compared to the second section 140. For example, a first outer cross-sectional dimension 136 of the first section 130 is greater than a second outer cross-sectional dimension 146 of a proximal portion of the second section 140, and the second outer cross-sectional dimension 146 is greater than a third outer cross-sectional dimension 156 of a distal portion of the second section 140. The outer cross-sectional dimension can decrease continuously and/or stepwise from one section to another distally along the support member 122.

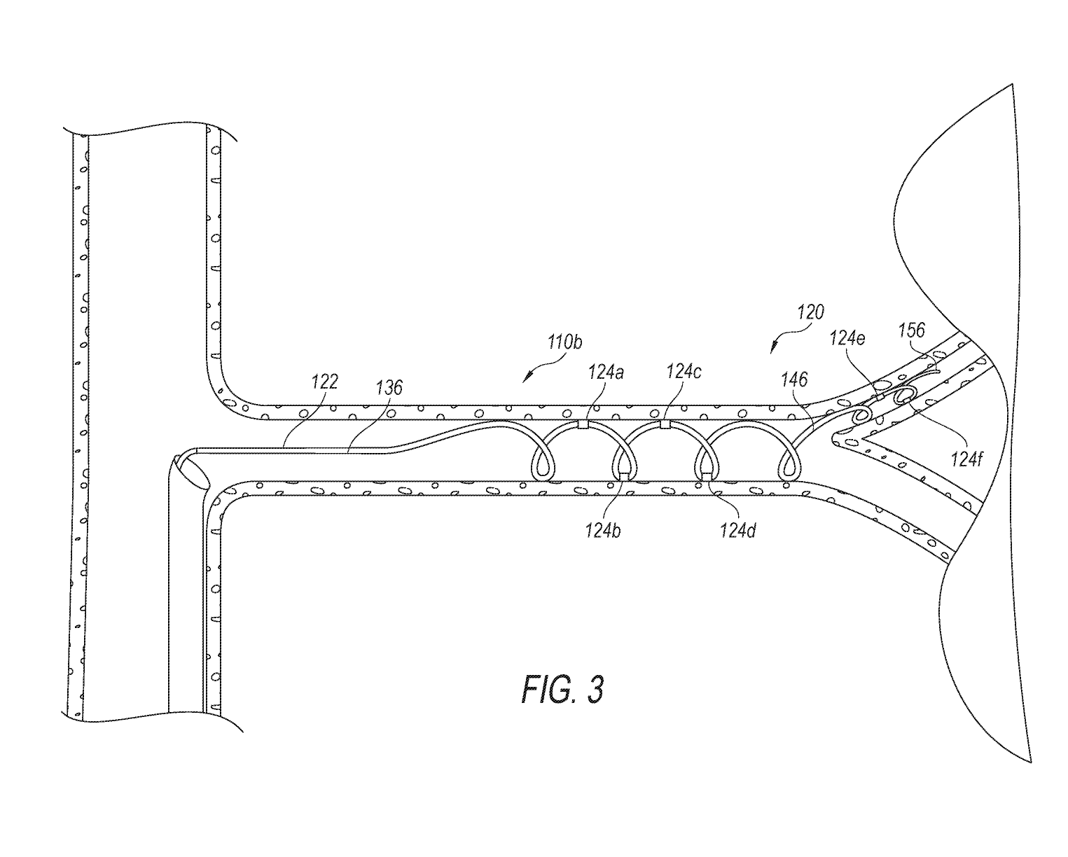

In some embodiments, as illustrated in FIG. 3, the first outer cross-sectional dimension 136 is sized and shaped to facilitate formation of an expanded state that accommodates an inner diameter of a typical renal artery (e.g., about 2-10 mm), and the second outer cross-sectional dimension 146 sized and shaped to facilitate formation of an expanded state that accommodates an inner diameter of a distal vascular portion, such as the distal vascular portion of the renal artery (e.g., about 0.25-5 mm). The third outer cross-sectional dimension 156 can be sized and shaped to facilitate formation of an expanded state that accommodates the inner diameter more distally along distal vascular portion (e.g., about 0.1-2 mm). In other embodiments, the support member 122 can have other outer cross-sectional dimensions depending on the body lumen within which it is configured to be deployed. The outer cross-sectional dimensions described herein can be measures of cross-sectional areas, diameters, transverse dimensions or other measures of size.

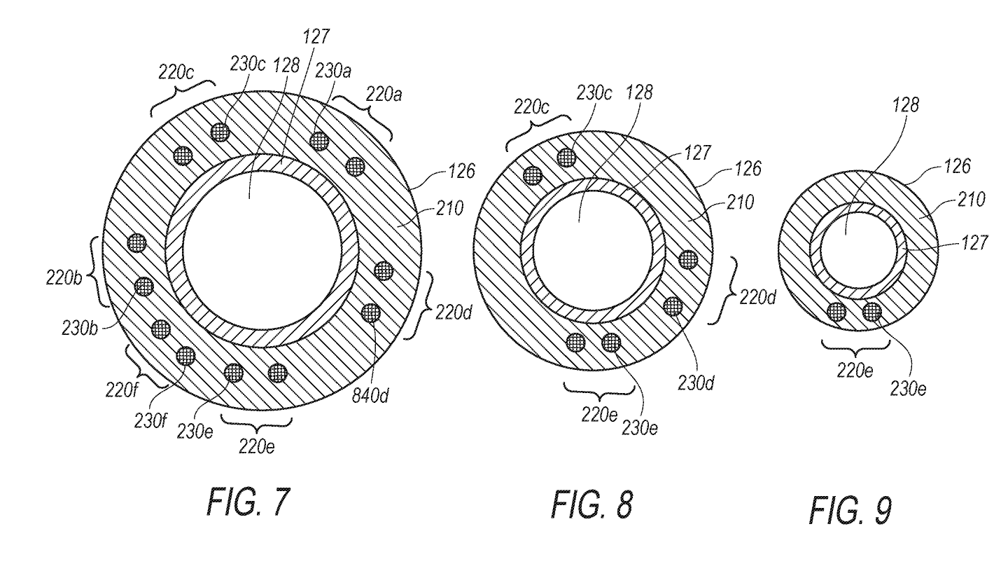

The support member 122 can comprise a wall 126 (e.g., a circumferential wall), a hollow or tubular core 127 within the wall 126, a lumen 128 within the core 127, and an atraumatic tip 138. See e.g. FIGS. 7-9. The lumen 128 can extend distally from a proximal opening in the handle 108 or from a side port (not shown) along the shaft 110 through the support member 122. As illustrated in FIG. 1, the energy delivery elements 124 are positioned along the wall 126. The wall 126 can be made from one or more materials (e.g., metals and/or polymers) formed into different arrangements (e.g., a braid or a mesh) suitable for supporting the energy delivery elements 124. The wall 126 and/or the core 127 can include a shape-memory memory material, such as a nickel-titanium alloy, suitable for imparting a shape to the support member 122 when expanded (e.g., deployed state). In the illustrated embodiment, the core 127 includes a shape memory material, such as nitinol, that tends to form the support member 122 into a desired configuration within a vessel. In other embodiments, the shape memory material is integrated into a portion of the wall 126, e.g., a braided shape memory layer over-molded or encapsulated within a polymeric material. The lumen 128 is configured to receive a wire (e.g., a guidewire, a lead wire, etc.) or a plurality of such wires extending from the proximal portion 110a of the elongated shaft 110 into the neuromodulation assembly 120 at the distal portion 110b. In some embodiments, the neuromodulation assembly 120 can have a plurality of lumens and each lumen can be configured to carry a wire or plurality of wires (e.g., guidewire, lead wire, etc.). In one embodiment, the guidewire holds the support member 122 in a non-expanded state for delivery (e.g., linear, straight, etc.), and upon removing the guidewire the support member 122 expands into a desired shape (e.g., spiral) imparted by the shape-memory core 127. In other embodiments, the support member 122 can expand to a desired deployed shape upon releasing the support member 122 from being radially constrained within a sheath by withdrawing the sheath and/or pushing the support member 122.

In the embodiment illustrated in FIG. 1, the neuromodulation assembly 120 includes six energy delivery elements 124 (individually identified by reference numbers 124a-f). In other embodiments, the neuromodulation assembly 120 can include a different number of energy delivery elements 124 (e.g., 1, 2, 8, 12, etc.). One or more of the energy delivery elements 124 can be electrodes configured to deliver therapeutic levels of RF energy and/or other forms of electrical energy to the vessel wall to ablate the nerves proximate to the treatment site. The energy delivery elements 124 can be configured to deliver power either together or independently of each other in a monopolar fashion, either simultaneously or sequentially, and/or may deliver power between any desired combination of the elements in a bipolar fashion. In some embodiments, the energy delivery elements can be configured to deliver correspondingly less energy to nerves along a distal vascular portion to avoid damaging the increasingly fragile tissues. Additionally, a first therapeutic neuromodulation energy can be delivered to one set of the energy delivery elements 124 (e.g., energy deliver elements 124a-d), while a second therapeutic neuromodulation energy can be delivered to another set of energy delivery elements (e.g., energy delivery elements 124d-f). The second therapeutic neuromodulation energy can be less than the first therapeutic neuromodulation energy to protect smaller, more sensitive structures in the distal vascular portion. The energy delivery elements 124 can also be configured to deliver constant power and/or monitor impedance indicating whether the energy delivery element(s) 124 are contacting the vessel wall.

The energy delivery elements 124 are spaced apart from each other along a length of the support member 122. In the embodiment shown in FIG. 1, for example, energy deliver elements 124a-c are spaced apart from each other along the first section 130 and energy deliver elements 124d-f are spaced apart from each other along the second section 140. The energy delivery elements 124 can be arranged along the support member 122 in a variety of patterns. For example, in the expanded state the energy delivery elements 124 can be positioned in multiple planes and on multiple sites across the support member 122. The energy delivery elements 124 may be mounted about, adhered to, and/or woven into the wall 126. In several embodiments, the electrodes 124 can be formed of gold in accordance with electrodes of existing neuromodulation devices. Each energy delivery element 124 can be electrically coupled to a pair of lead wires. In each pair, a copper wire can be used for conducting electrical power for neuromodulation and a constantan wire can form a thermocouple at the energy delivery element 124 for temperature monitoring.

As illustrated in FIG. 1, the first energy delivery element 124a has a first cross-sectional dimension 125a and each of the other energy delivery elements 124b-f has a decrementally changing cross-sectional dimension 125b-f, respectively, in the distal direction. For example, the cross-sectional dimension 125a of the first energy delivery element 124a is greater than the cross-sectional dimension 125b of the second energy delivery element 124b, which is greater than the cross-sectional dimension 125c of the third energy delivery element 124c, and so on to the last energy delivery element 124f.

The outer cross-sectional dimension of the support member 122 can be related to the number of pairs of lead wires carried by the wall 126 at any particular longitudinal location. For example, the outer cross-sectional dimensions 136, 146 and 156 are partially determined by the number of lead wires carried by the wall 126. Each of the outer cross-sectional dimensions (e.g., 136, 146 and 156) relates to a combined cross-sectional area of the pairs of lead wires extending through the neuromodulation assembly 120 at each cross-sectional area (e.g., FIG. 7 shows six pairs at outer cross sectional dimension 136, FIG. 8 shows three pairs at second outer cross-sectional dimension 146, and FIG. 9 shows one pair at the third outer cross sectional dimension 156). As such, the number of pairs of lead wires carried by the support member 122 decreases by one pair from energy delivery element 124a to energy delivery element 124b, from energy delivery element 124b to energy delivery element 124c, and so on to energy delivery element 124f.

One or more sensors (not shown), such as one or more temperature (e.g., thermocouple, thermistor, etc.), impedance, pressure, optical, flow, chemical, and/or other sensors, may be located proximate to, within, or integral with the energy delivery elements 124. The sensor(s) and the energy delivery elements 124 can be connected to one or more supply wires or optical fibers (not shown) that transmit signals from the sensor(s).

In other embodiments, the neuromodulation device 120 can include other electrodes, transducers, or other elements to deliver energy to modulate nerves using other suitable neuromodulation modalities, such as pulsed electrical energy, microwave energy, optical energy, ultrasound energy (e.g., intravascularly delivered ultrasound, and/or high-intensity focused ultrasound (HIFU)), direct heat energy, radiation (e.g., infrared, visible, and/or gamma radiation), and/or other suitable types of energy. For example, some of the energy delivery elements 124 can be defined by radiation emitters that expose target nerves to radiation at a wavelength that causes a previously administered photosensitizer to react, such that it damages or disrupts the nerves. The radiation emitters can be optical elements coupled to fiber optic cables (e.g., extending through the elongated shaft 110) for delivering radiation from a radiation source (e.g., an energy generator) at an extracorporeal location to the target tissue at the vessel, or may be internal radiation sources (e.g., LEDs) that are electrically coupled to a power source at an extracorporeal location via electrical leads within the elongated shaft 110. In embodiments where one or more of the energy delivery elements 124 are defined by radiation emitters, a photosensitizer (e.g., oxytetracycline, a suitable tetracycline analog, and/or other suitable photosensitive compounds that preferentially bind to neural tissue) can be administered to a patient (e.g., orally, via injection, through an intravascular device, etc.), and preferentially accumulate at selected nerves (e.g., rather than other tissues proximate to the selected nerves). For example, more of the photosensitizer can accumulate in perivascular nerves around a blood vessel than in the non-neural tissues of the blood vessel. The mechanisms for preferentially accumulating the photosensitizer at the nerves can include faster uptake by the nerves, longer residual times at the nerves, or a combination of both. After a desired dosage of the photosensitizer has accumulated at the nerves, the photosensitizer can be irradiated using energy delivery elements 124. The energy delivery elements 124 can deliver radiation to the target nerves at a wavelength that causes the photosensitizer to react such that it damages or disrupts the nerves. For example, the photosensitizer can become toxic upon exposure to the radiation. Because the photosensitizer preferentially accumulates at the nerves and not the other tissue proximate the nerves, the toxicity and corresponding damage is localized primarily at the nerves. This form of irradiative neuromodulation can also or alternatively be incorporated in any one of the neuromodulation assemblies described herein. Further details and characteristics of neuromodulation assemblies with radiation emitters are included in U.S. patent application Ser. No. 13/826,604, which is incorporated herein by reference in its entirety.

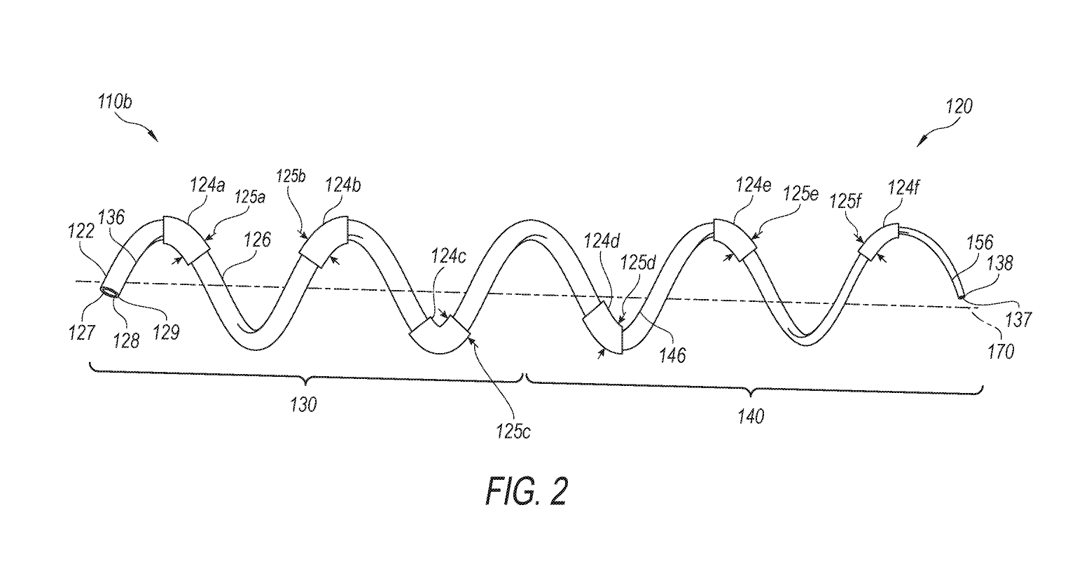

FIG. 2 is a isometric view of the distal portion 110b of the neuromodulation assembly 120 in an expanded state in accordance with an embodiment of the present technology. In the illustrated embodiment, the support member 122 has a spiral shape imparted to both the first section 130 and the second section 140 by the shape memory core 127. The spiral shape of the support member 122 extends around an imaginary longitudinal central axis 170 and extends from an imaginary cross-sectional plane into three dimensions. The spiral shape is imparted by the core 127 after the guidewire is removed (e.g., retracted) from the lumen 128 of the distal portion 110b of the elongated shaft 110. In other embodiments, the shape can be imparted on at least one other portion of the support member 122.

FIG. 3 is a side view of the distal portion 110b of a neuromodulation device positioned within both a proximal vascular portion (e.g., a main renal artery) and a distal vascular portion (e.g., a renal branch vessel in this example) in accordance with an embodiment of the present technology. In operation, the neuromodulation assembly 120 is intravascularly delivered to a treatment site within both the proximal vascular portion vessel and the distal vascular portion in a single act of positioning the neuromodulation assembly 120. As illustrated, the support member 122 extends from the proximal vascular portion into the distal vascular portion and is designed to apply a desired outward radial force to the vessel walls when deployed and expanded into the spiral shape. The deployed support member 122 can be configured and positioned such that the energy delivery elements 124a-d are pressed against the interior wall of the proximal vascular portion and the energy delivery elements 124e and 124f are pressed against the interior wall of the distal vascular portion. In other embodiments, the neuromodulation assembly 120 is positioned such that fewer of energy delivery elements 124 contact the proximal vascular portion, such as energy delivery elements 124a-c, while more of the energy delivery elements 124d-f contact the distal vascular portion. The energy delivery elements 124 deliver therapeutic neuromodulation energy to nerves proximate to the treatment site(s) at the interior wall(s) of the blood vessel(s), which is expected to modulate or ablate nerves at the treatment site (e.g., both efferent renal nerves and afferent renal nerves) and/or proximate to the treatment site.

Several embodiments of the neuromodulation assembly 120 shown in FIGS. 1-3 are expected to provide efficient, enhanced neuromodulation in anatomical structures with different sizes. Since the first section 130 of the neuromodulation assembly 120 is configured to deliver energy to areas of a proximal vascular portion having a first structure (e.g., a main renal artery) and the second section 140 is configured to deliver energy to areas of a distal vascular portion having a second structure (e.g., a more distal portion of the main renal artery or a renal branch vessel) that would otherwise not be accessible by a device suitable for only the first structure of the proximal vascular portion, the neuromodulation assembly 120 is expected to enhance the ablation of nerves adjacent the different vascular structure, and in particular renal nerves. Moreover, several embodiments of the neuromodulation assembly 120 enable such dual neuromodulation in a single deployment using a single device. This saves time and reduces challenges with positioning and repositioning devices in the renal vasculature.

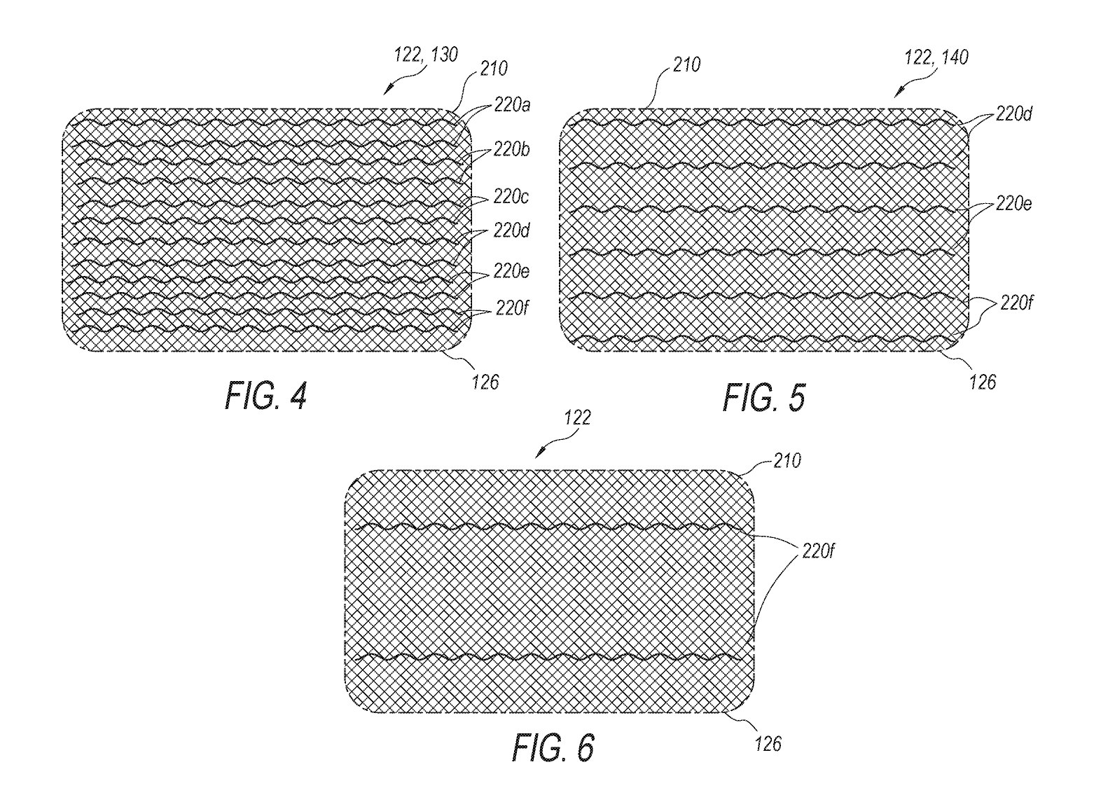

FIGS. 4-6 are enlarged views of lead wire pairs 220 (identified individually by reference numbers 220a-f in FIG. 4) along the wall 126 at different locations along the support member 122 identified by encircled areas FIG. 4, FIG. 5 and FIG. 6 shown in FIG. 1. Instead of running the lead wire pairs 220 through the lumen (FIG. 1) of the elongated shaft 110 (FIG. 1), the lead wire pairs 220 are carried by the wall 126. For example, referring to FIG. 4 and FIG. 1 together, the proximal-most portion of the first section 130 of the neuromodulation assembly 120 just proximal the first energy delivery element 124a carries all six of the lead wire pairs 220a-f. Referring to FIG. 5 and FIG. 1 together, the proximal-most portion of the second section 140 of the neuromodulation assembly 120 just proximal to fourth energy delivery element 124d carries lead wire pairs 220d-f, and referring to FIG. 6 and FIG. 1 together the portion of the neuromodulation assembly 120 just proximal the sixth energy delivery element 124f carries one lead wire pair 220f. Each lead wire pair 220a-f includes two lead wires that are coupled to a corresponding one of the energy delivery elements 124a-f (FIG. 1), respectively. In the illustrated embodiments, the lead wire pairs 220 are incorporated into the wall 126 by braiding (e.g., weaving) each of the lead wires into a braided structure 210 incorporated into the wall 126. In other embodiments, the lead wire pairs 220 can be disposed on, or otherwise incorporated into the wall 126 using a different technique and/or form of material. The lead wire pairs 220 of the embodiments described herein are insulated, but in other embodiments the lead wire pairs 220 may not be insulated. The illustrated number of lead wire pairs 220 is not intended to be limiting, and additional pairs of lead wire pairs coupled to additional energy delivery elements or sensors (e.g., temperature or impedance sensors) can be carried by the wall 126 in other embodiments. However, the number of lead wire pairs 220 is related to the maximum outer cross-sectional dimension of the second section 140 of the neuromodulation assembly 120 suitable for delivery into the distal vascular portion.

FIGS. 7-9 are partially schematic enlarged cross-sectional views of the distal portion of the neuromodulation device of FIG. 1 taken along cross-sections 7-7, 8-8 and 9-9 of FIG. 1, respectively, that show additional features of lead wire pairs 220. The neuromodulation assembly 120 can include various features generally similar to those of the neuromodulation assembly 120 described above with reference to FIGS. 1-6, and like reference numbers refer to common components in FIGS. 1-9. For example, FIG. 7 illustrates the lead wire pairs 220a-f integrated into one portion of the wall 126 at a proximal portion of the support member 122, and FIGS. 8 and 9 show cross-sections progressively more distally along the support member 122 with progressively fewer lead wire pairs 220. The lead wires 220 can be braided into a braided structure 210 of the wall 126 as explained above with reference to FIGS. 4-6, or the lead wire pairs 220 can be in lumens or channels extending through the wall 126 with each lead wire in a separate lumen or a plurality of lead wires in a single lumen.

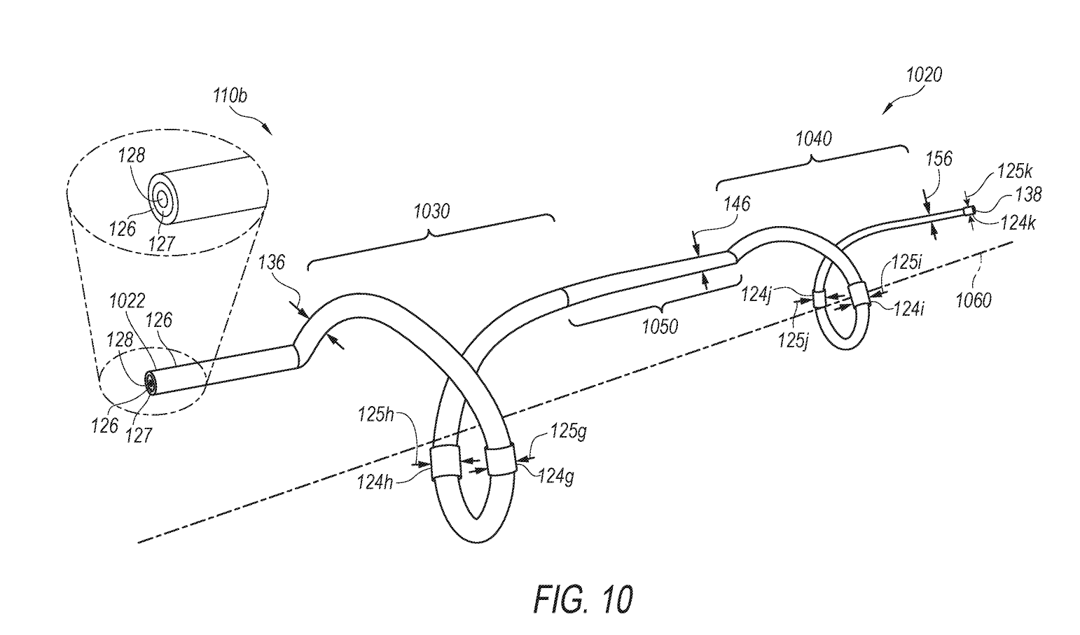

FIG. 10 is a partially schematic enlarged isometric view of a distal portion of a neuromodulation assembly 1020 in accordance with another embodiment of the present technology. The neuromodulation assembly 1020 can include various features generally similar to those described above with reference to FIGS. 1-9, and like reference numbers refer to like components in FIGS. 1-10. In this embodiment, the neuromodulation assembly 1020 has a support structure 1022 including a first section 1030, a second section 1040, and an intermediate section 1050 between the first and second sections 1030 and 1040. In the embodiment shown in FIG. 10, the first section 1030 of the support member 1022 is configured to have a first shape (e.g. spiral), the second section 1040 of the support member 1022 is configured to have a second shape (e.g., spiral), and the intermediate section 1050 between the first and second sections 1030 and 1040 can have third shape different than the first and second shapes. The intermediate section 1050, for example, can be at least substantially linear. The shapes of the first section 1030, second section 1040 and intermediate section 1050 can be imparted to the support member 122 by the core 127 following at least partial removal (e.g., retraction) of a guidewire from the lumen 128 as the neuromodulation assembly 120 is being deployed. The spiral shape of the first section 1030 and the second section 1040 extends around an imaginary longitudinal central axis 1060 and extends from an imaginary transverse plane into three dimensions. Alternatively, each section of the support member 1022 can have a different shape. Shapes other than those illustrated and described herein can be imparted to the support member 1022 in an expanded state.

The first section 1030 can be sized and shaped to accommodate positioning and deployment in a first vascular portion having an inner diameter of, e.g., about 2-10 mm, and the second section 1040 is sized and shaped to be positioned in a second vascular portion having an inner diameter of, e.g., about 0.25-5 mm or about 0.1-2 mm. The outer cross-sectional dimension of the support member 1022 decreases (e.g., constantly or step-wise) distally, and in particular the outer cross-sectional dimension of the second section 1040 is less than that of the first section 1030. As described above, the outer cross-sectional dimension(s) (e.g., 136, 146 and 156) are partially based on a cross-sectional dimension of a pair, or number of pairs, of lead wire pairs 220 carried by the wall 126 of the support member 1022.

As illustrated in FIG. 10, energy delivery elements 124g-j are disposed along a length of the support member 1022. Although FIG. 10 illustrates two energy delivery elements 124g and 124h located in the first section 1030 and two energy delivery elements 124i and 124j located in the second section 1040, additional energy delivery elements can be disposed within the first section 1030 and/or the second section 1040. As described above, the energy delivery elements 124g-j each have a cross-sectional dimension 125g-j, respectively, that decrementally changes distally along the support member 1022. In the illustrated embodiment, the neuromodulation assembly 1020 includes an energy delivery element 124k disposed at the tip 138 having a cross-sectional dimension 125k. Alternatively, the tip 138 can be an atraumatic tip.

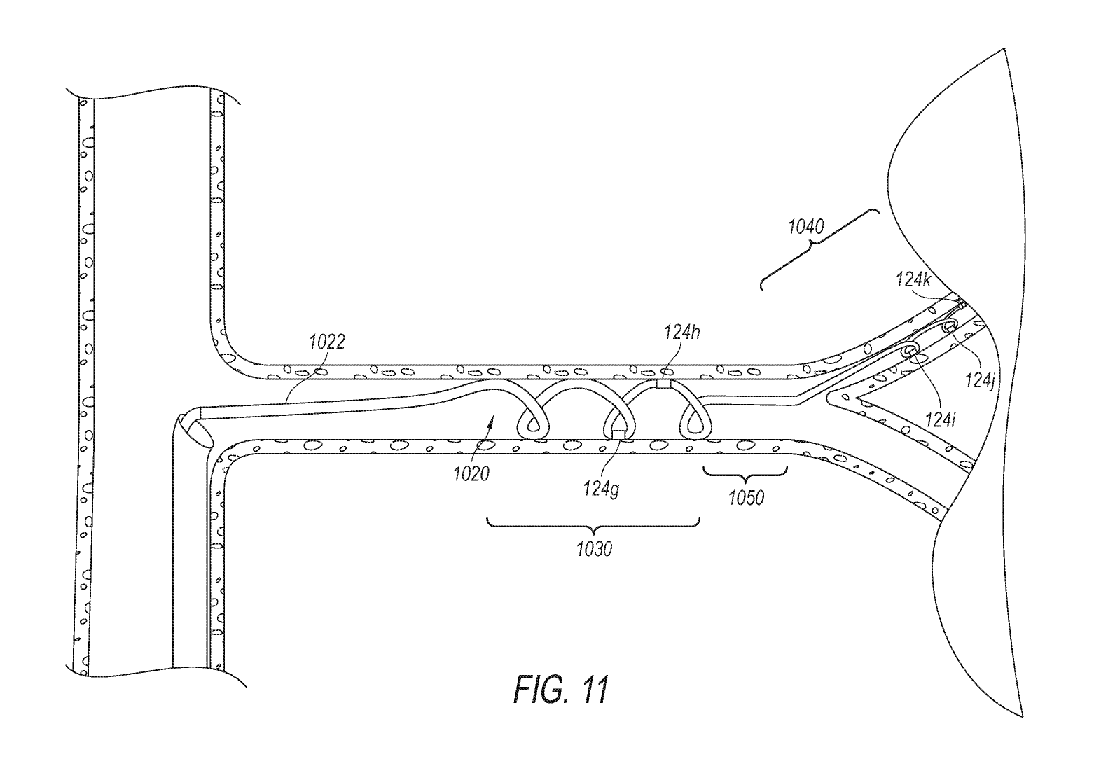

FIG. 11 is a side view of the neuromodulation assembly 1020 of FIG. 10 positioned within a blood vessel in accordance with an embodiment of the present technology. In the illustrated embodiment, the neuromodulation assembly 1020 is expanded and deployed in the proximal vascular portion and the distal vascular portion at the same time. As a result, the neuromodulation assembly 1020 can be intravascularly delivered to at least two treatment sites having different structures (e.g., different diameters) in a single deployment of the neuromodulation assembly 1020. This allows the energy delivery element 124g to be positioned to deliver energy to ablate nerves along the proximal vascular portion (e.g., a portion of the main renal artery) of the patient, whereas the energy delivery element 124i is positioned to deliver energy to ablate nerves along the distal vascular portion (e.g., an anterior branch of the renal artery of the patient). Similarly, the energy delivery element 124k can be positioned to deliver energy to ablate renal nerves even more distally along the vessel. In other embodiments, the energy delivery element 124 can be positioned differently than shown in FIG. 11. The intermediate section 1050 can be positioned at a distal portion of the main renal artery and extend to or through a proximal portion of the branch vessel to support positioning of the first section 1030 and the second section 1040 at the desired locations in the proximal vascular portion (e.g., main renal artery) and the distal vascular portion (e.g., branch vessel), respectively.

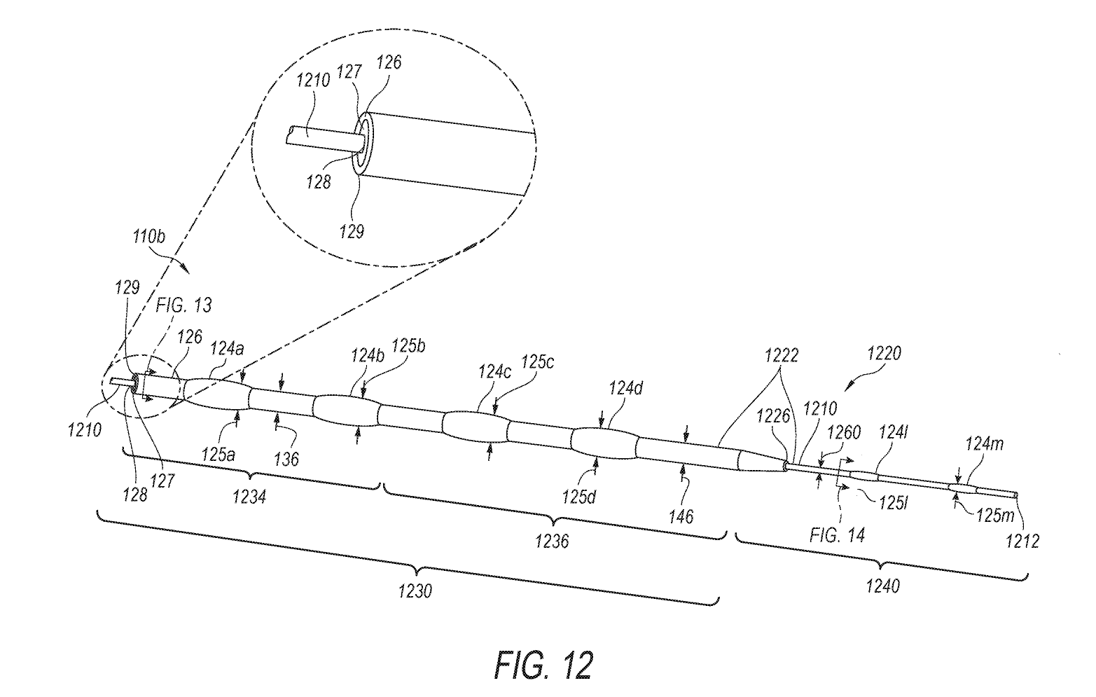

FIG. 12 is an isometric view of a distal portion of a neuromodulation assembly 1220 with an enlarged view of one portion of the neuromodulation assembly 1220 in accordance with yet another embodiment of the present technology. The neuromodulation assembly 1220 can include a number of features generally similar to those described above with reference to FIGS. 1-11, and like reference numbers refer to similar components in FIGS. 1-12. In this embodiment, however, the neuromodulation assembly 1220 has a support member 1222 including a first section 1230 and a second section 1240 that is slidably received within the first section 1230. For example, the first section 1230 of the support member 1222 is defined by a portion of the distal end 110b (FIG. 1) of the elongated shaft 110 (FIG. 1), and the second section 1240 is defined by a distal portion of a guidewire 1210 that passes through the elongated shaft 110. The guidewire 1210 can thus be advanced or retracted while positioning the neuromodulation assembly 1220 within the patient's vessel. The illustrated guidewire 1210 has an atraumatic tip 1212, and in some embodiments, the atraumatic tip 1212 can have a shape (e.g., a hockey tip) configured to direct (e.g., steer) the neuromodulation assembly 1220 during positioning inside the patient's vessel lumen.

The first section 1230 of the neuromodulation assembly 1220 can have an outer cross-sectional dimension that decreases (e.g., constant or step-wise) from the proximal end of the first section 1230 to the distal end of the first section 1230. For example, the first section 1230 of the neuromodulation assembly 1220 can itself have a proximal portion 1232 with a first diameter 136 and a distal portion 1234 with a second diameter 146 less than the first diameter. In addition, the second section 1240 (e.g., the distal portion of the guidewire 1210) has a third outer cross-sectional dimension 1260 less than the second outer cross-sectional dimension 146. In the illustrated embodiment, the first outer cross-sectional dimension 136 can be 0.09 inches, and the third outer cross-sectional dimension 1260 can be 0.014 inches (e.g., 36 mm). As described above, the outer cross-sectional dimensions can be measures of cross-sectional areas, diameters, transverse dimensions or other dimensions of the neuromodulation assembly 120. In other embodiments, the first outer cross-sectional dimension 136, the second outer cross-sectional dimension 146, and the third outer cross-sectional dimension 1260 can each be less than 0.09 inches or greater than 0.09 inches up to 0.20 inches.

In the illustrated embodiment, energy delivery elements 124a-d are disposed along the first section 1230 and energy delivery elements 124l and 124m are disposed along the second section 1240. In other embodiments, each section can be configured to carry only a single energy delivery element, or alternatively a plurality of energy delivery elements. Similar to other embodiments of the neuromodulation assembly 120 described herein, energy delivery elements 124a-d each have a cross-sectional dimension 125a-d, respectively, and distal energy delivery elements 124l-m each have a cross-sectional dimension 125l-m, respectively. Cross-sectional dimensions 125a-b can each be greater than cross-sectional dimensions 125c-d. In some embodiments, cross-sectional dimensions 125a-d are the same, or they can decrementally change (e.g., constant or step-wise) along the axial length of the support member 122. For example, cross-sectional dimension 125a is greater than cross-sectional dimension 125b, which is greater than cross-sectional dimension 125c, which is greater than cross-sectional dimension 125d.

FIG. 12 illustrates the neuromodulation assembly 120 in a linear (e.g., straight) configuration for delivery into the patient's vessel(s). Similar to the other embodiments of the neuromodulation assemblies 120 and 1020 described above with respect to FIGS. 1-11, a shape (e.g., helical, spiral, linear, etc.), or a plurality of shapes, can be imparted to the neuromodulation assembly 1220 of FIG. 12 when expanded. In some embodiments, the shape can be imparted to each section (first section 1230 and second section 1240) of the support member 1222 independently of the other sections. The core 127 in the first section 1230 of the support member 1222 can include a shape memory material, e.g., nitinol, configured to impart the desired shape to the first section 1230 upon expansion. Additionally, a portion of the guidewire 1210 can be formed from a shape memory material, e.g., nitinol. For example, the distal portion of the guidewire 1210 with the energy delivery elements 124l and 124m can be made from a shape-memory material, while the more proximal portion of the guidewire 1210 can be an elastic material without shape memory. Since the guidewire 1210 can be made from a combination of shape-memory material and elastic non-memory material, the neuromodulation assembly 1220 can be retained in the straight position shown in FIG. 12 by a sheath.

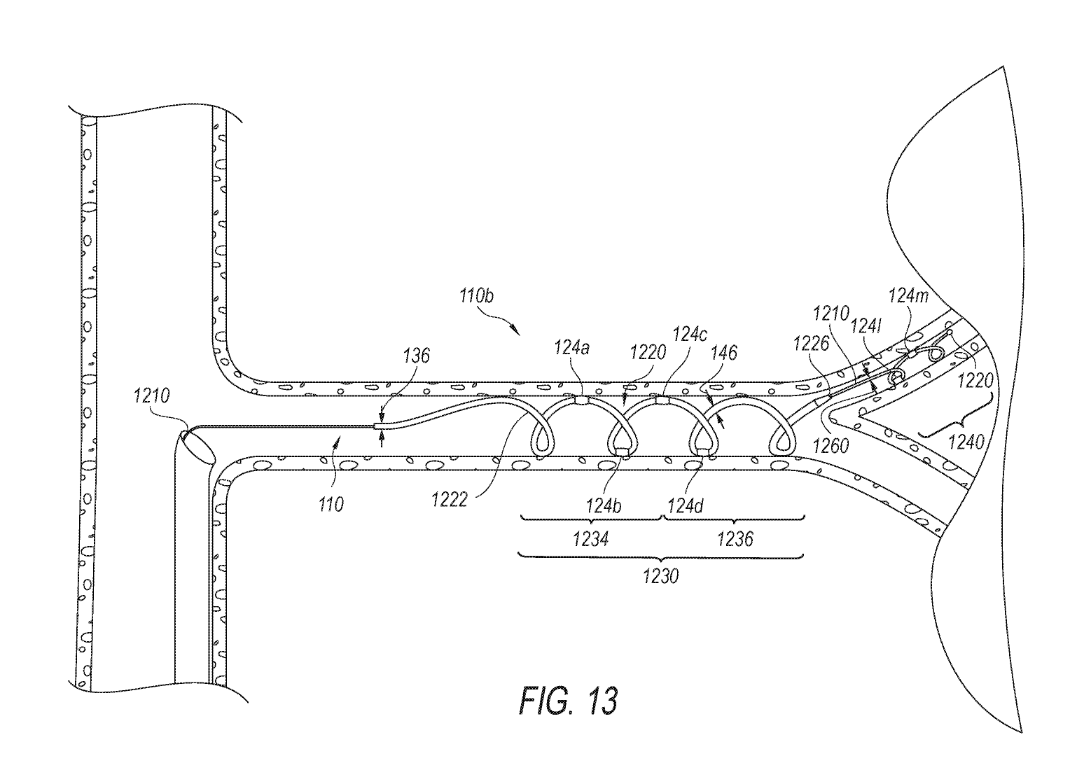

FIG. 13 is a side view of the neuromodulation assembly 1220 of FIG. 12 in a deployed state within the vasculature of a patient. In operation, the neuromodulation assembly 1220 is positioned so that the distal end of the first section 1230 is located at or near the transition from a proximal vascular portion (e.g., the main renal artery) to a distal vascular portion (e.g., a branch of main the renal artery) while the neuromodulation assembly 1220 is within a sheath. The guidewire 1210 is then advanced from the through a distal opening 1226 of the first section 1230. In this embodiment, the guidewire 1210 extends from the distal opening 1226 and into the distal vascular portion (e.g., branch of the main renal artery) where it forms into a spiral or other suitable shape for pressing the energy delivery elements 124l and 124m against the wall of the distal vascular portion. The first section 1230 of the neuromodulation assembly 1220 is then deployed by withdrawing the sheath (not shown). The neuromodulation assembly 1220 can accordingly be positioned at a plurality of treatment sites having different sizes and shapes in a single deployment (e.g., at the same time). For example, when positioned, the first section 1230 is expanded into the deployed state at one treatment site and the second section 1240 is expanded at another treatment site by releasing the guidewire 1210 from the lumen 128. In other embodiments, other suitable positioning methods and deployment methods can be used to position and deploy the neuromodulation assembly 1220.

In the embodiment shown in FIG. 13, the first section 1230 of the deployed support member 1222 has a spiral shape configured to press the energy delivery elements 124a-d against the interior wall of the proximal vascular portion (e.g., main renal artery) to contact the first treatment site. The second section 1240 of the support member 1222 also has a spiral shape configured to press the energy delivery elements 124l and 124m against the interior wall of the distal vascular portion to contact the second treatment site. The first section 1230 is designed to apply the desired outward radial force to the proximal vascular portion, whereas the second section 1240 is designed to apply the desired outward radial force to the distal vascular portion. The desired outward radial force applied to the proximal vascular portion can be greater than the desired outward radial force applied to the distal vascular portion to avoid damaging the distal vascular portion by applying too much outward radial force.

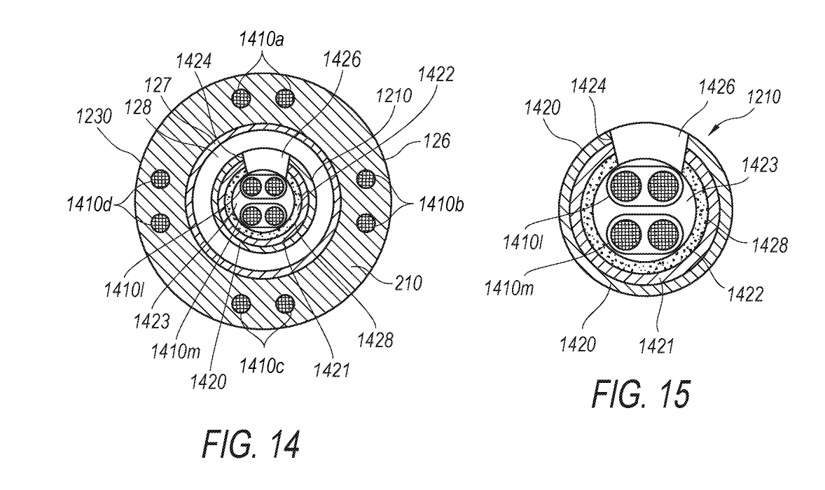

The energy delivery elements 124a-d and 124l-m in FIG. 13 are each electrically coupled to lead wire pairs 1410a-d and 1410l-m, respectively, shown in FIGS. 14 and 15. The lead wire pairs 1410a-d can be carried by the first section 1230 of the support member 1222, and the lead wire pairs 1410l-m can be carried by the second section 1240 (e.g., the guidewire 1210) of the support member 1222. In certain embodiments, the energy delivery elements 124a-d can deliver a first therapeutic neuromodulation energy to nerves proximate to a first treatment site in the proximal vascular portion, and the energy deliver elements 124l-m can deliver a second therapeutic neuromodulation energy to the nerves proximate to a second treatment site in the distal vascular portion. The second therapeutic neuromodulation energy can be less than the first therapeutic neuromodulation energy to avoid damaging the distal vascular portion, nerves distal from the second treatment site, and/or adjacent tissue.

FIGS. 14 and 15 are partially schematic enlarged cross-sectional views of the distal portion of the neuromodulation device of FIG. 12 taken along cross-sections 14-14 and 15-15 of FIG. 12. For example, FIG. 14 illustrates a cross-sectional view of the neuromodulation assembly 1220 at the first outer cross-sectional dimension 136 (FIG. 13). In this embodiment, the guidewire 1210 extends through at least a portion of the lumen 128 of the neuromodulation assembly 1220. As illustrated, the guidewire 1210 has an outer layer 1420, a middle layer 1421, an inner layer 1422 (e.g., silver nickel), and a lumen 1423. The outer layer 1420 is formed of cobalt chromium for push/pull axial strength along the guidewire 1210. The middle layer 1421 is formed of tantalum for radiopacity. In the illustrated embodiment, the lumen 1423 of the guidewire 1210 is formed by cutting a slot 1426 having sidewalls 1424 through the outer, middle and inner layers 1420, 1421, 1422 along at least a portion of the length of the guidewire 1210 and then etching a central portion of the remaining material of the inner layer 1422. The slot 1426 can be formed using a laser, and the central region of the inner layer 1422 can be removed using a chemical etching process (e.g., a liquid chemical etch). In some embodiments, the layers can be formed using any material, or materials, suitable to perform at least similar functions of those described above. The lumen 1423 is sized and shaped to retain lead wires coupled to energy delivery elements disposed on the guidewire 1210. As illustrated, two lead wire pairs 1410l and 1410m are in the lumen 1423, and the lead wire pairs 1410l and 1410m are electrically coupled to energy delivery elements 124l and 124m disposed at the third section 1250 (e.g., the distal portion of the guidewire 1210). In other embodiments, the lumen 1423 can be sized and shaped to retain a number of pairs of lead wires sufficient to electrically couple to each energy delivery element 124 disposed on the guidewire 1210.

FIG. 15 illustrates a cross-sectional view of the neuromodulation assembly at the third outer cross-sectional dimension 1260. The third outer cross-sectional dimension 1260 is distal to the distal opening 1226 (FIG. 12) and includes the guidewire 1210. The outer cross-sectional dimension of the guidewire 1210 shown in FIG. 15 is therefore smaller than an outer cross-sectional dimension of the first portion 1230 shown in FIG. 14. Similar to FIG. 14, the lumen 1423 of the guidewire contains the two pairs of lead wires 1410l and 1410m.

Although the embodiments of the neuromodulation assemblies 120, 1020 and 1220 shown in FIGS. 1-15 have partially spiral/helically-shaped configurations and partially linear configurations, spiral/helically-shaped configurations, or linear configurations, in other embodiments, the neuromodulation assemblies can have other suitable shapes, sizes, and/or configurations. For example, in further embodiments, the distal portion 110b of the elongated shaft 110 (FIG. 1) can have other suitable shapes (e.g., semi-circular, curved, straight, etc.), and/or the neuromodulation assemblies can include multiple support members configured to carry one or more energy delivery elements 124, e.g., electrodes. Other suitable devices and technologies are described in, for example, U.S. patent application Ser. No. 12/910,631, filed Oct. 22, 2010; U.S. patent application Ser. No. 13/279,205, filed Oct. 21, 2011; U.S. patent application Ser. No. 13/279,330, filed Oct. 23, 2011; U.S. patent application Ser. No. 13/281,360, filed Oct. 25, 2011; U.S. patent application Ser. No. 13/281,361, filed Oct. 25, 2011; PCT Application No. PCT/US11/57754, filed Oct. 25, 2011; U.S. Provisional Patent Application No. 61/646,218, filed May 5, 2012; U.S. patent application Ser. No. 13/793,647, filed Mar. 11, 2013; U.S. Provisional Patent Application No. 61/961,874, filed Oct. 24, 2013; and U.S. patent application Ser. No. 13/670,452, filed Nov. 6, 2012. All of the foregoing applications are incorporated herein by reference in their entireties. Non-limiting examples of devices and systems include the Symplicity Flex™ and the Symplicity Spyral™ multielectrode RF ablation catheter.

### II. Selected Examples of Neuromodulation Devices and Related Systems

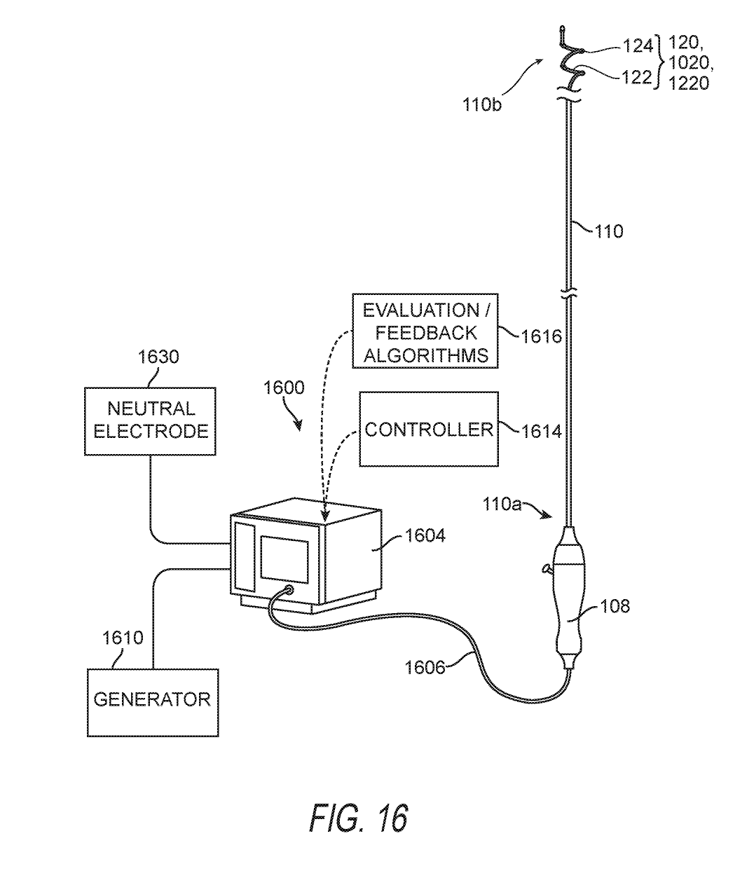

FIG. 16 is a partially schematic illustration of a therapeutic system 1600 (“system 1600”) configured in accordance with an embodiment of the present technology. The system 1600 can be used in conjunction with embodiments of the neuromodulation assemblies 120, 1020 and 1220 described above with references to FIGS. 1-15 to deliver neuromodulation therapy. In addition, the system 1600 can be used to provide the autonomic modulation therapy of the methods described herein.

As shown in FIG. 16, the system 1600 can include a neuromodulation device 1602 that includes the handle 108, the elongated shaft 110 having a distal portion 110b configured to be positioned at a target site within the renal artery, and any of the neuromodulation assemblies 120, 1020 and 1220 described above. In some embodiments, the elongated shaft 110, at least two energy delivery elements 124, and at least two pairs of lead wires 220 are integral components of a single catheter (not shown) configured for delivering neuromodulation energy to treatment sites in both a proximal vascular portion and a distal vascular portion that have different sizes and/or shapes. In other embodiments, elongated shaft 110, at least two energy delivery elements 124, and at least two pairs of lead wires 220 can be components of more than one catheter (not shown). In the illustrated embodiment, the system 1600 can further include a console 1604 and a cable 1606 extending between the console 1604 and the handle 108. The console 1604 can be configured to control, monitor, supply, and/or otherwise support operation of the neuromodulation device 1602. In addition, the console 1604 can be configured to provide feedback to an operator before, during, and/or after a treatment procedure via an evaluation/feedback algorithm 1616.

The console 1604 can further be configured to generate a selected form and/or magnitude of energy for delivery to tissue at the treatment site via the neuromodulation assemblies 120, 1020 and 1220, and therefore the console 1604 may have different configurations depending on the treatment modality of the neuromodulation device 1602. For example, when the neuromodulation device 1602 is configured for electrode-based, heat-element-based, or transducer-based treatment, the console 1604 can include an energy generator 1610 (shown schematically) configured to generate radio frequency (RF) energy (e.g., monopolar and/or bipolar RF energy), pulsed energy, microwave energy, optical energy, ultrasound energy (e.g., intravascularly delivered ultrasound and/or high-intensity focused ultrasound (HIFU)), direct heat energy, radiation (e.g., infrared, visible, and/or gamma radiation), and/or another suitable type of energy. In this configuration, the console 1604 can also include evaluation/feedback algorithms 1616 for controlling energy delivery elements 124 of neuromodulation assemblies 120, 1020 and 1220. If the impedance changes during energy delivery, the power can be adjusted at the generator 1610. In selected embodiments, the generator 1610 can be configured to deliver a monopolar electric field via one or more of the energy delivery elements 124. In such embodiments, a neutral or dispersive electrode 1630 may be electrically coupled to the generator 1610 and attached to the exterior of the patient. When the neuromodulation device 1602 is configured for cryotherapeutic treatment, the console 1604 can include a refrigerant reservoir (not shown), and can be configured to supply the neuromodulation device 1602 with refrigerant. Similarly, when the neuromodulation device 1602 is configured for chemical-based treatment (e.g., drug infusion), the console 1604 can include a chemical reservoir (not shown) and can be configured to supply the neuromodulation device 1602 with one or more chemicals.

In various embodiments, the system 1600 can further include a controller 1614 communicatively coupled to the neuromodulation device 1602. The controller 1614 can be configured to initiate, terminate, and/or adjust operation of one or more components (e.g., the energy delivery elements 124) of the neuromodulation device 1602 directly and/or via the console 1604 and/or via a wired or wireless communication link. In various embodiments, the system 1600 can include multiple controllers. In other embodiments, the neuromodulation device 1602 can be communicatively coupled to a single controller 1614. The controller(s) 1614 can be integrated with the console 1604 or the handle 108 positioned outside the patient and used operate the system 1600. In other embodiments, the controller 1614 can be omitted or have other suitable locations (e.g., within the handle 108, along the cable 1606, etc.). The controller 1614 can include computer-implemented instructions to initiate, terminate, and/or adjust operation of one or more components of the neuromodulation device 1602 directly and/or via another aspect of the system (e.g., the console or handle 108). For example, the controller 1614 can further provide instructions to the neuromodulation device 1602 to apply neuromodulatory energy to the treatment site (e.g., RF energy via the energy delivery elements 124). The controller 1614 can be configured to execute an automated control algorithm and/or to receive control instructions from an operator. Further, the controller 1614 can include or be linked to the evaluation/feedback algorithm 1616 that can provide feedback to an operator before, during, and/or after a treatment procedure via a console, monitor, and/or other user interface.

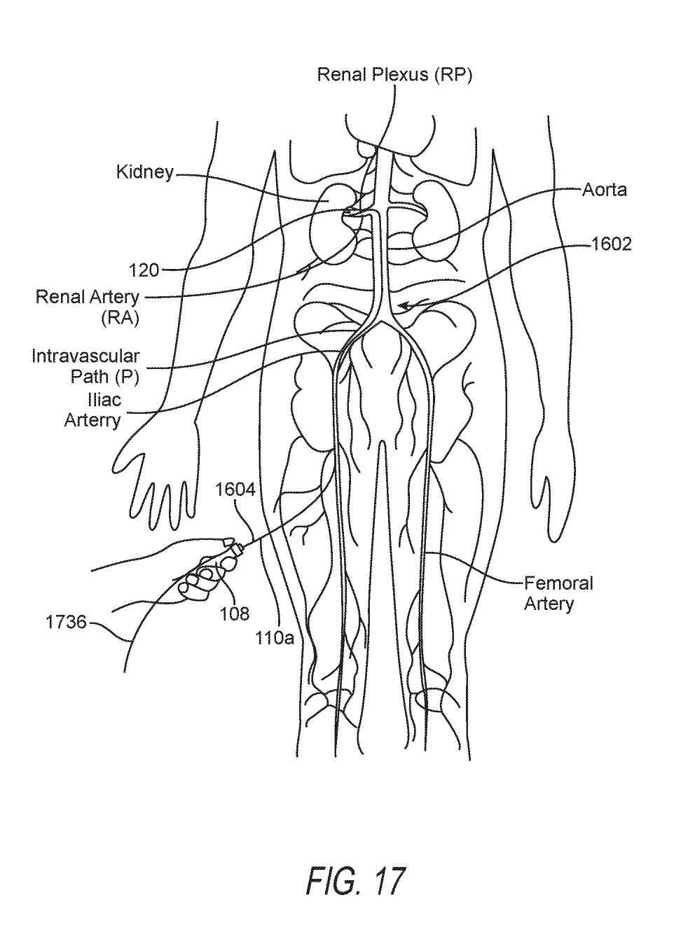

FIG. 17 (with additional reference to FIG. 16) illustrates modulating renal nerves in accordance with an embodiment of the system 1600. The neuromodulation device 1602 provides access to the renal plexus RP through an intravascular path P, such as a percutaneous access site in the femoral (illustrated), brachial, radial, or axillary artery to a targeted treatment site within a respective renal artery RA. By manipulating the proximal portion 110a of the elongated shaft 110 from outside the intravascular path P, a clinician may advance the elongated shaft 110 through the sometimes tortuous intravascular path P and remotely manipulate the distal portion 110b (FIG. 16) of the elongated shaft 110. In the embodiment illustrated in FIG. 17, the neuromodulation assembly 120 is delivered intravascularly to the treatment site using a guidewire 1736 in an OTW technique. At the treatment site, the guidewire 1736 can be at least partially withdrawn or removed, and the neuromodulation assembly 120 can transform or otherwise be moved to a deployed arrangement for recording neural activity and/or delivering energy at the treatment site. In other embodiments, the neuromodulation assembly 120 may be delivered to the treatment site within a guide sheath (not shown) with or without using the guidewire 1736. When the neuromodulation assemblies 120, 1020 and 1220 are at the treatment site, the guide sheath may be at least partially withdrawn or retracted and the neuromodulation assemblies 120, 1020 and 1220 can be transformed into the deployed arrangement. In other embodiments, the elongated shaft 110 may be steerable itself such that the neuromodulation assemblies 120, 1020 and 1220 may be delivered to the treatment site without the aid of the guidewire 1736 and/or guide sheath.

Image guidance, e.g., computed tomography (CT), fluoroscopy, intravascular ultrasound (IVUS), optical coherence tomography (OCT), intracardiac echocardiography (ICE), or another suitable guidance modality, or combinations thereof, may be used to aid the clinician's positioning and manipulation of the neuromodulation assemblies 120, 1020 and 1220. For example, a fluoroscopy system (e.g., including a flat-panel detector, x-ray, or c-arm) can be rotated to accurately visualize and identify the target treatment site. In other embodiments, the treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering the neuromodulation assemblies 120, 1020 and 1220. Further, in some embodiments, image guidance components (e.g., IVUS, OCT) may be integrated with the neuromodulation device 1602 and/or run in parallel with the neuromodulation device 1602 to provide image guidance during positioning of neuromodulation assemblies 120, 1020 and 1220. For example, image guidance components (e.g., IVUS or OCT) can be coupled to neuromodulation assemblies 120, 1020 and 1220 to provide three-dimensional images of the vasculature proximate the treatment site to facilitate positioning or deploying the multi-electrode assembly within the target renal vascular structure.

Energy from the energy delivery elements 124 may then be applied to target tissue to induce one or more desired neuromodulating effects on localized regions of the renal artery RA and adjacent regions of the renal plexus RP, which lay intimately within, adjacent to, or in close proximity to the adventitia of the renal artery RA. The purposeful application of the energy may achieve neuromodulation along all or at least a portion of the renal plexus RP. The neuromodulating effects are generally a function of, at least in part, power, time, contact between the energy delivery elements and the vessel wall, and blood flow through the vessel. The neuromodulating effects may include denervation, thermal ablation, and/or non-ablative thermal alteration or damage (e.g., via sustained heating and/or resistive heating). Desired thermal heating effects may include raising the temperature of target neural fibers above a desired threshold to achieve non-ablative thermal alteration, or above a higher temperature to achieve ablative thermal alteration. For example, the target temperature may be above body temperature (e.g., approximately 37° C.) but less than about 45° C. for non-ablative thermal alteration, or the target temperature may be about 45° C. or higher for the ablative thermal alteration. Desired non-thermal neuromodulation effects may include altering the electrical signals transmitted in a nerve.

Hypothermic effects may also provide neuromodulation. For example, a cryotherapeutic applicator may be used to cool tissue at a treatment site to provide therapeutically-effective direct cell injury (e.g., necrosis), vascular injury (e.g., starving the cell from nutrients by damaging supplying blood vessels), and sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Embodiments of the present technology can include cooling a structure at or near an inner surface of a renal artery wall such that proximate (e.g., adjacent) tissue is effectively cooled to a depth where sympathetic renal nerves reside. For example, the cooling structure is cooled to the extent that it causes therapeutically effective, cryogenic renal-nerve modulation. Sufficiently cooling at least a portion of a sympathetic renal nerve is expected to slow or potentially block conduction of neural signals to produce a prolonged or permanent reduction in renal sympathetic activity.

### III. Renal Neuromodulation

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic over activity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, chemically-induced, or induced in another suitable manner or combination of manners at one or more suitable treatment sites during a treatment procedure. The treatment site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

Renal neuromodulation can include a cryotherapeutic treatment modality alone or in combination with another treatment modality. Cryotherapeutic treatment can include cooling tissue at a treatment site in a manner that modulates neural function. For example, sufficiently cooling at least a portion of a sympathetic renal nerve can slow or potentially block conduction of neural signals to produce a prolonged or permanent reduction in renal sympathetic activity. This effect can occur as a result of cryotherapeutic tissue damage, which can include, for example, direct cell injury (e.g., necrosis), vascular or luminal injury (e.g., starving cells from nutrients by damaging supplying blood vessels), and/or sublethal hypothermia with subsequent apoptosis. Exposure to cryotherapeutic cooling can cause acute cell death (e.g., immediately after exposure) and/or delayed cell death (e.g., during tissue thawing and subsequent hyperperfusion). Neuromodulation using a cryotherapeutic treatment in accordance with embodiments of the present technology can include cooling a structure proximate an inner surface of a body lumen wall such that tissue is effectively cooled to a depth where sympathetic renal nerves reside. For example, in some embodiments, a cooling assembly of a cryotherapeutic device can be cooled to the extent that it causes therapeutically-effective, cryogenic renal neuromodulation. In other embodiments, a cryotherapeutic treatment modality can include cooling that is not configured to cause neuromodulation. For example, the cooling can be at or above cryogenic temperatures and can be used to control neuromodulation via another treatment modality (e.g., to protect tissue from neuromodulating energy).

Renal neuromodulation can include an electrode-based or transducer-based treatment modality alone or in combination with another treatment modality. Electrode-based or transducer-based treatment can include delivering electricity and/or another form of energy to tissue at a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at a treatment location. For example, neuromodulation in accordance with embodiments of the present technology can include delivering RF energy, pulsed energy, microwave energy, optical energy, focused ultrasound energy (e.g., high-intensity focused ultrasound energy), or another suitable type of energy alone or in combination. An electrode or transducer used to deliver this energy can be used alone or with other electrodes or transducers in a multi-electrode or multi-transducer array. Furthermore, the energy can be applied from within the body (e.g., within the vasculature or other body lumens in a catheter-based approach) and/or from outside the body (e.g., via an applicator positioned outside the body). Furthermore, energy can be used to reduce damage to non-targeted tissue when targeted tissue adjacent to the non-targeted tissue is subjected to neuromodulating cooling.

Neuromodulation using focused ultrasound energy (e.g., high-intensity focused ultrasound energy) can be beneficial relative to neuromodulation using other treatment modalities. Focused ultrasound is an example of a transducer-based treatment modality that can be delivered from outside the body. Focused ultrasound treatment can be performed in close association with imaging (e.g., magnetic resonance, computed tomography, fluoroscopy, ultrasound (e.g., intravascular or intraluminal), optical coherence tomography, or another suitable imaging modality). For example, imaging can be used to identify an anatomical position of a treatment location (e.g., as a set of coordinates relative to a reference point). The coordinates can then entered into a focused ultrasound device configured to change the power, angle, phase, or other suitable parameters to generate an ultrasound focal zone at the location corresponding to the coordinates. The focal zone can be small enough to localize therapeutically-effective heating at the treatment location while partially or fully avoiding potentially harmful disruption of nearby structures. To generate the focal zone, the ultrasound device can be configured to pass ultrasound energy through a lens, and/or the ultrasound energy can be generated by a curved transducer or by multiple transducers in a phased array (curved or straight).

Heating effects of electrode-based or transducer-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of vascular or luminal structures that perfuse the target neural fibers. In cases where vascular structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of vascular or luminal structures that perfuse the target fibers. In some patients, it can be desirable to heat tissue to temperatures that are sufficient to ablate the target neural fibers or the vascular or luminal structures, but that are less than about 90° C. (e.g., less than about 85° C., less than about 80° C., or less than about 75° C.).

Renal neuromodulation can include a chemical-based treatment modality alone or in combination with another treatment modality. Neuromodulation using chemical-based treatment can include delivering one or more chemicals (e.g., drugs or other agents) to tissue at a treatment location in a manner that modulates neural function. The chemical, for example, can be selected to affect the treatment location generally or to selectively affect some structures at the treatment location over other structures. The chemical, for example, can be guanethidine, ethanol, phenol, a neurotoxin, or another suitable agent selected to alter, damage, or disrupt nerves. A variety of suitable techniques can be used to deliver chemicals to tissue at a treatment location. For example, chemicals can be delivered via one or more needles originating outside the body or within the vasculature or other body lumens. In an intravascular example, a catheter can be used to intravascularly position a therapeutic element including a plurality of needles (e.g., micro-needles) that can be retracted or otherwise blocked prior to deployment. In other embodiments, a chemical can be introduced into tissue at a treatment location via simple diffusion through a body lumen wall, electrophoresis, or another suitable mechanism. Similar techniques can be used to introduce chemicals that are not configured to cause neuromodulation, but rather to facilitate neuromodulation via another treatment modality.

### IV. Related Anatomy and Physiology

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

A. The Sympathetic Chain

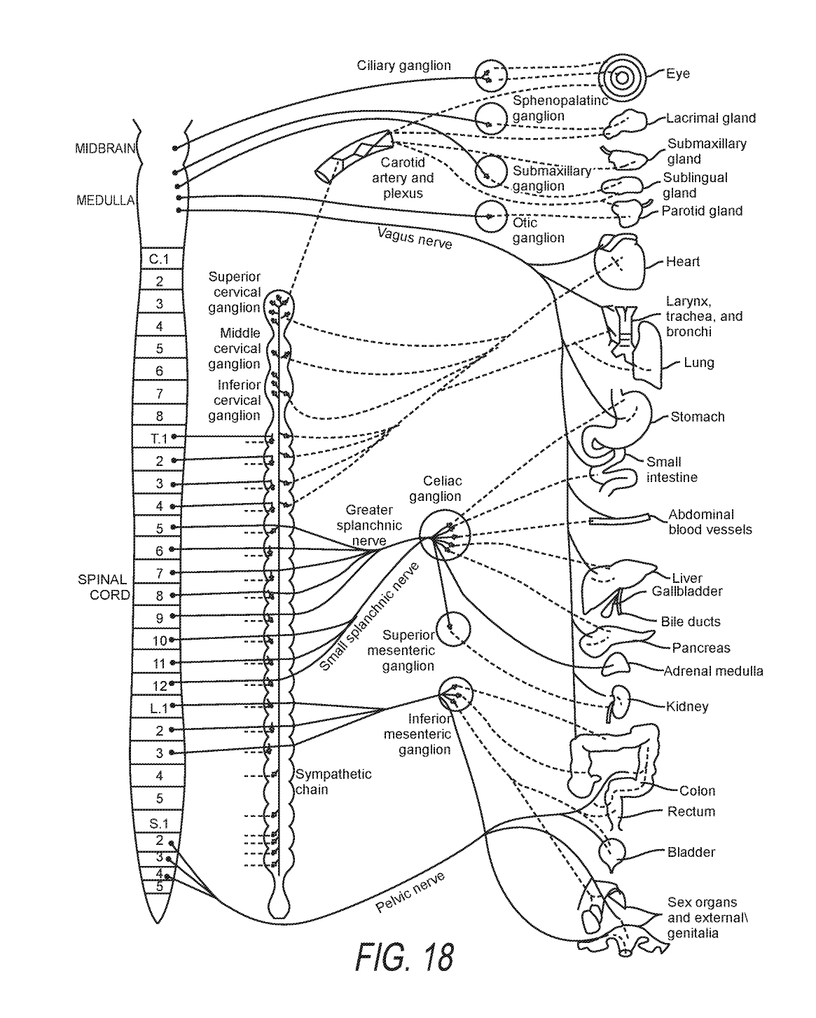

As shown in FIG. 18, the SNS provides a network of nerves that allows the brain to communicate with the body. Sympathetic nerves originate inside the vertebral column, toward the middle of the spinal cord in the intermediolateral cell column (or lateral horn), beginning at the first thoracic segment of the spinal cord and are thought to extend to the second or third lumbar segments. Because its cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

1. Innervation of the Kidneys

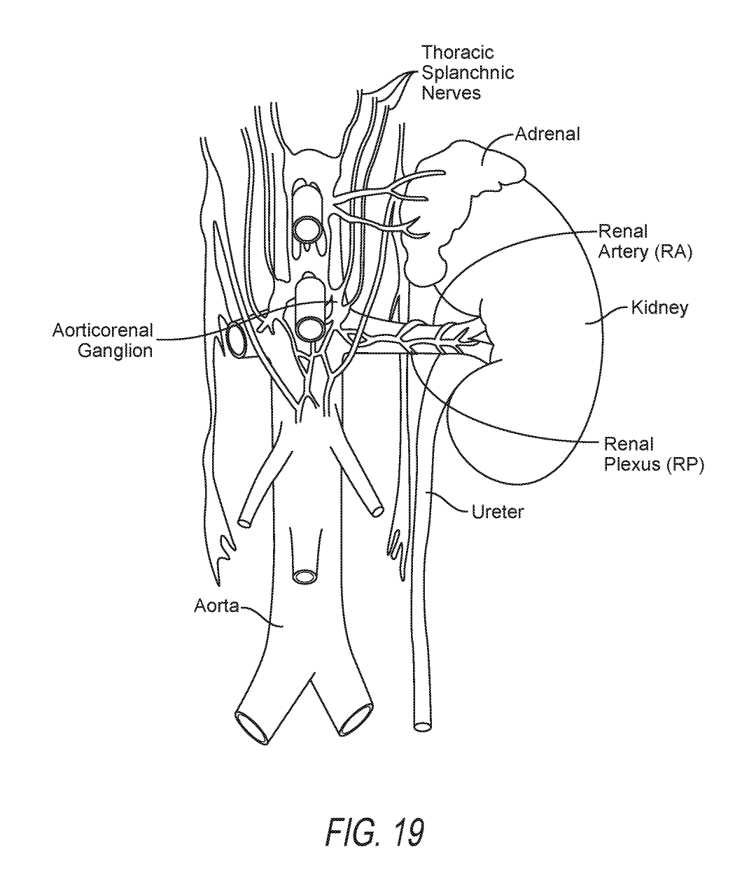

As FIG. 19 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, first lumbar splanchnic nerve, second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

2. Renal Sympathetic Neural Activity

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); and raise blood pressure. Afferent messages carry signals from various organs and sensory receptors in the body to other organs and, particularly, the brain.

Hypertension, heart failure and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease are characterized by heightened sympathetic nervous activation. In patients with end stage renal disease, plasma levels of norepinephrine above the median have been demonstrated to be predictive for both all-cause death and death from cardiovascular disease. This is also true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

(i) Renal Sympathetic Efferent Activity

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation is likely a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release) and diuretics (intended to counter the renal sympathetic mediated sodium and water retention). However, the current pharmacologic strategies have significant limitations including limited efficacy, compliance issues, side effects and others.

(ii) Renal Sensory Afferent Nerve Activity

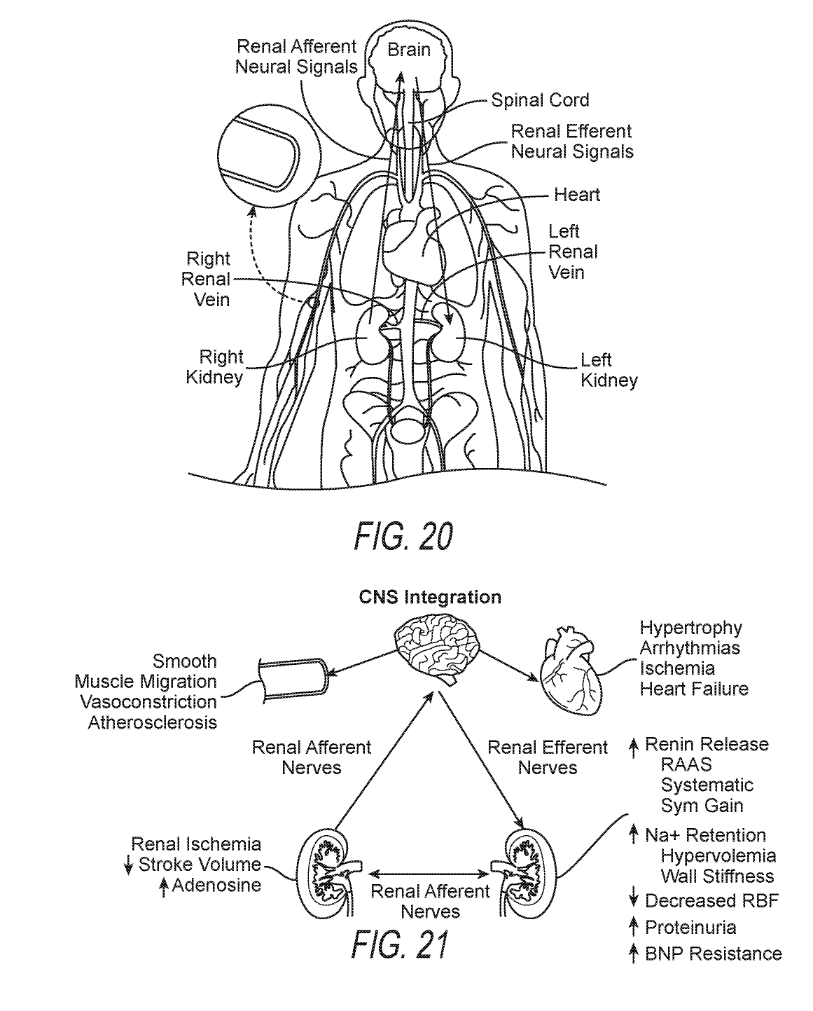

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 20 and 21, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

B. Additional Clinical Benefits of Renal Denervation

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 18. For example, as previously discussed, a reduction in central sympathetic drive may reduce the insulin resistance that afflicts people with metabolic syndrome and Type II diabetics. Additionally, patients with osteoporosis are also sympathetically activated and might also benefit from the down regulation of sympathetic drive that accompanies renal denervation.

C. Achieving Intravascular Access to the Renal Artery

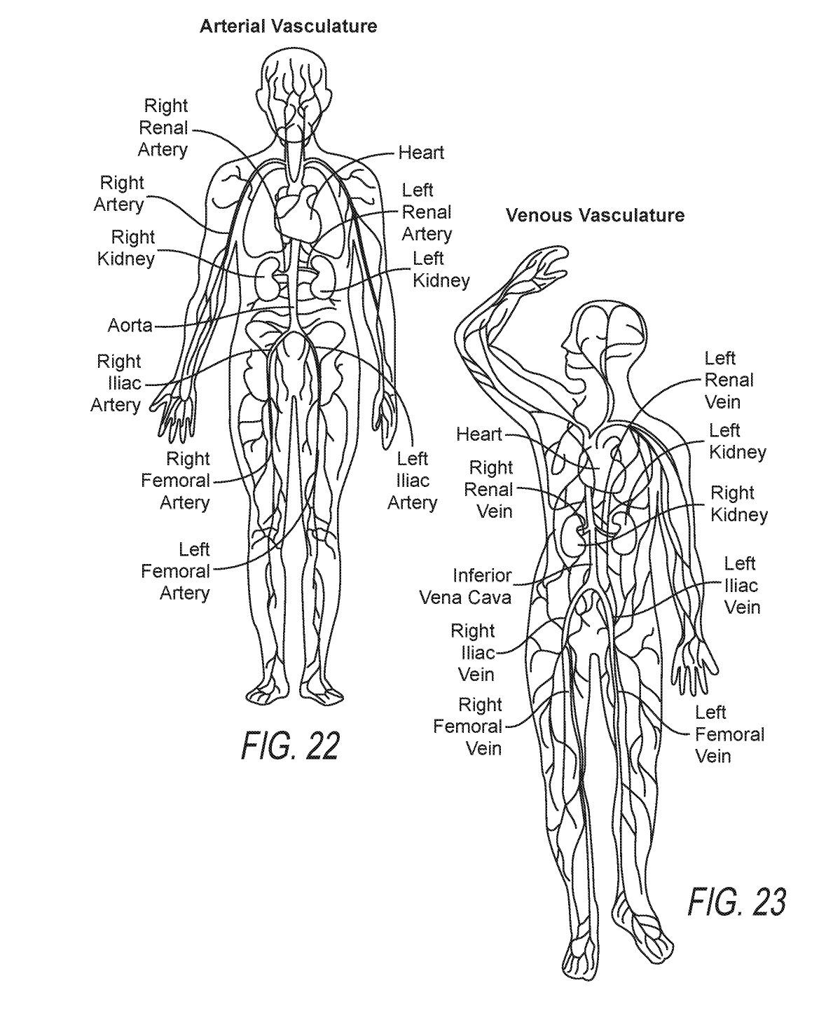

In accordance with the present technology, neuromodulation of a left and/or right renal plexus (RP), which is intimately associated with a left and/or right renal artery, may be achieved through intravascular access. As FIG. 22 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 23 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique.

D. Properties and Characteristics of the Renal Vasculature

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, etc. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e. cause the wall of the artery to pulse).

Even after accessing a renal artery and facilitating stable contact between neuromodulatory apparatus and a luminal surface of the artery, nerves in and around the adventia of the artery should be safely modulated via the neuromodulatory apparatus. Effectively applying thermal treatment from within a renal artery is non-trivial given the potential clinical complications associated with such treatment. For example, the intima and media of the renal artery are highly vulnerable to thermal injury. As discussed in greater detail below, the intima-media thickness separating the vessel lumen from its adventitia means that target renal nerves may be multiple millimeters distant from the luminal surface of the artery. Sufficient energy should be delivered to or heat removed from the target renal nerves to modulate the target renal nerves without excessively cooling or heating the vessel wall to the extent that the wall is frozen, desiccated, or otherwise potentially affected to an undesirable extent. A potential clinical complication associated with excessive heating is thrombus formation from coagulating blood flowing through the artery. Given that this thrombus may cause a kidney infarct, thereby causing irreversible damage to the kidney, thermal treatment from within the renal artery should be applied carefully. Accordingly, the complex fluid mechanics and thermodynamic conditions present in the renal artery during treatment, particularly those that may impact heat transfer dynamics at the treatment site, may be important in applying energy (e.g., heating thermal energy) and/or removing heat from the tissue (e.g., cooling thermal conditions) from within the renal artery.

The neuromodulatory apparatus should also be configured to allow for adjustable positioning and repositioning of the energy delivery element within the renal artery since location of treatment may also impact clinical efficacy. For example, it may be tempting to apply a full circumferential treatment from within the renal artery given that the renal nerves may be spaced circumferentially around a renal artery. In some situations, a full-circle lesion likely resulting from a continuous circumferential treatment may be potentially related to renal artery stenosis. Therefore, the formation of more complex lesions along a longitudinal dimension of the renal artery and/or repositioning of the neuromodulatory apparatus to multiple treatment locations may be desirable. It should be noted, however, that a benefit of creating a circumferential ablation may outweigh the potential of renal artery stenosis or the risk may be mitigated with certain embodiments or in certain patients and creating a circumferential ablation could be a goal. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging. Manipulation of a device in a renal artery should also consider mechanical injury imposed by the device on the renal artery. Motion of a device in an artery, for example by inserting, manipulating, negotiating bends and so forth, may contribute to dissection, perforation, denuding intima, or disrupting the interior elastic lamina.

Blood flow through a renal artery may be temporarily occluded for a short time with minimal or no complications. However, occlusion for a significant amount of time should be avoided because to prevent injury to the kidney such as ischemia. It could be beneficial to avoid occlusion all together or, if occlusion is beneficial to the embodiment, to limit the duration of occlusion, for example to 2-5 minutes.

Based on the above described challenges of (1) renal artery intervention, (2) consistent and stable placement of the treatment element against the vessel wall, (3) effective application of treatment across the vessel wall, (4) positioning and potentially repositioning the treatment apparatus to allow for multiple treatment locations, and (5) avoiding or limiting duration of blood flow occlusion, various independent and dependent properties of the renal vasculature that may be of interest include, for example, (a) vessel diameter, vessel length, intima-media thickness, coefficient of friction, and tortuosity; (b) distensibility, stiffness and modulus of elasticity of the vessel wall; (c) peak systolic, end-diastolic blood flow velocity, as well as the mean systolic-diastolic peak blood flow velocity, and mean/max volumetric blood flow rate; (d) specific heat capacity of blood and/or of the vessel wall, thermal conductivity of blood and/or of the vessel wall, and/or thermal convectivity of blood flow past a vessel wall treatment site and/or radiative heat transfer; (e) renal artery motion relative to the aorta induced by respiration, patient movement, and/or blood flow pulsatility; and (f) the take-off angle of a renal artery relative to the aorta. These properties will be discussed in greater detail with respect to the renal arteries. However, dependent on the apparatus, systems and methods utilized to achieve renal neuromodulation, such properties of the renal arteries, also may guide and/or constrain design characteristics.

As noted above, an apparatus positioned within a renal artery should conform to the geometry of the artery. Renal artery vessel diameter, D_{RA}, typically is in a range of about 2-10 mm, with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >5 mm from inner wall of the renal artery) to avoid non-target tissue and anatomical structures such as the renal vein.

An additional property of the renal artery that may be of interest is the degree of renal motion relative to the aorta induced by respiration and/or blood flow pulsatility. A patient's kidney, which is located at the distal end of the renal artery, may move as much as 4″ cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

### V. Additional Examples

The following examples are illustrative of several embodiments of the present technology:

1. A neuromodulation device, comprising:
an elongated shaft having a distal portion with a cross-sectional area and a distal end, the cross-sectional area decreasing along a length of the distal portion toward the distal end;
a neuromodulation assembly at the distal portion of the elongated shaft, the neuromodulation assembly comprising a plurality of energy delivery elements longitudinally spaced along the distal portion of the elongated shaft, wherein the plurality of energy delivery elements comprises at least a first electrode and a second electrode distal to the first electrode, and wherein the second electrode has a cross-sectional area less than that of the first electrode; and
a plurality of pairs of lead wires carried by a portion of the elongated shaft, each pair of lead wires coupled to an energy delivery element,
wherein the distal portion of the shaft is sized and shaped for delivery into a distal vascular portion of renal vasculature in a human.

2. The neuromodulation device of example 1 wherein the elongated shaft further comprises a braided circumferential wall, and wherein the lead wires are integrated with the braided circumferential wall.

3. The neuromodulation device of example 1 or example 2 wherein the cross-sectional area changes decrementally along the distal portion of the distal portion of the elongated shaft, and wherein the decrement relates to a combined cross-sectional area of a pair of the plurality of lead wires.

4. The neuromodulation device of any one of examples 1 to 3 wherein the distal portion of the elongated shaft comprises a greater quantity of lead wires at the first electrode than at the second electrode.

5. A neuromodulation device for delivering neuromodulation therapy in a vessel of a human patient, the neuromodulation device comprising:
an elongated shaft having a distal portion with a first section and a second section distal to the first section, the first section having a first outer cross-sectional dimension and the second section having a second outer cross-sectional dimension less than the second outer cross-sectional dimension;
a neuromodulation assembly at the distal portion of the elongated shaft, the neuromodulation assembly comprising—
   a first energy delivery element at the first section of the elongated shaft; and
   a second energy delivery element at the second section of the elongated shaft.

6. The neuromodulation device of example 5, further comprising:
a first pair of lead wires electrically coupled to the first energy delivery element; and
a second pair of lead wires electrically coupled to the second energy delivery element.

7. The neuromodulation device of example 5 or example 6 wherein:
the first energy delivery element is a first electrode having a first outer cross-sectional dimension; and
the second energy delivery element is a second electrode having a second outer cross-sectional dimension less than the first outer cross-sectional dimension of the first electrode.

8. The neuromodulation device of any one of examples 5 to 7 wherein the elongated shaft comprises a braid.

9. The neuromodulation device of any one of examples 5 to 8 wherein a first pair and a second pair of lead wires are incorporated into the braid.

10. The neuromodulation device of any one of examples 5 to 9 wherein the distal portion of the elongated shaft includes a shaped portion having a helical shape when the distal portion is in an expanded state.

11. The neuromodulation device of any one of examples 5 to 10 wherein the first section is a first shaped portion and the second section is a second shaped portion, and wherein the first and second shaped portions are helical when the distal portion is in an expanded state.

12. The neuromodulation device of any one of examples 5 to 11 wherein the first and second shaped sections are separated by a linear portion.

13. The neuromodulation device of any one of examples 5 to 12 wherein the elongated shaft comprises a guidewire lumen terminating in an opening at the distal portion of the elongated shaft, and wherein the neuromodulation device further comprises:
a guidewire configured to extend through the guidewire lumen and through the opening; and
a guidewire energy delivery element positioned at a distal section of the guidewire.

14. The neuromodulation device of example 13 wherein the guidewire further comprises at least one pair of lead wires electrically coupled to the guidewire energy delivery element.

15. The neuromodulation device of example 13 wherein the distal section of the guidewire defines the second section of the distal portion of the elongated shaft.

16. The neuromodulation device of example 13 wherein the guidewire has an outer cross-sectional dimension of 0.36 mm (0.014 inch).

17. The neuromodulation device of any one of examples 5 to 16, further comprising an atraumatic tip at the distal portion of the elongated shaft.

18. The neuromodulation device of any one of examples 5 to 17 wherein the distal portion of the elongated shaft has a distal end, and wherein the neuromodulation assembly further comprises a third energy delivery element at the distal end.

19. The neuromodulation device of any one of examples 5 to 18 wherein the elongated shaft further comprises a pre-formed nitinol core configured to impart a shape to the distal portion of the elongated shaft when the distal portion is in an expanded state.

20. The neuromodulation device of any one of examples 5 to 19 wherein the elongated shaft, the first energy delivery element, the second energy delivery element, the first pair of lead wires, and the second pair of lead wires are integral components of a single catheter.

21. The neuromodulation device of any one of examples 5 to 20 wherein the second energy delivery element is configured to deliver energy to ablate renal nerves along a distal vascular portion of a renal vascular structure of the patient.

22. The neuromodulation device of any one of examples 5 to 21 wherein the first outer cross-sectional dimension is a first outer diameter, and wherein the second outer cross-sectional dimension is a second outer diameter.

23. The neuromodulation device of any one of examples 5 to 22 wherein:
the first and second sections of the distal portion of the elongated shaft are two of a plurality of sections of the distal portion of the elongated shaft;
the first and second energy delivery elements are two of a plurality of energy delivery elements positioned at corresponding sections of the distal portion of the elongated shaft; and
each section and each energy delivery element have an decrementally changing outer cross-sectional dimension as the sections are positioned closer to a distal end of the elongated shaft.

24. A method of administering neuromodulation therapy to a patient, the method comprising:
delivering a distal portion of a neuromodulation device to a treatment site within a proximal vascular portion and a distal vascular portion of a renal vascular structure of a human, the distal portion of the neuromodulation device having a plurality of energy delivery elements spaced along a length of the distal portion, wherein the distal portion of the neuromodulation device has a first outer cross-sectional dimension at a first section of the distal portion and a second outer cross-sectional dimension at a second section of the distal portion, wherein the second section is distal to the first section, and wherein the first outer cross-sectional dimension is greater than the second outer cross-sectional dimension; and
delivering neuromodulation energy to renal nerves within the proximal vascular structure and/or the distal vascular structure via at least one energy delivery element of the neuromodulation device.

25. The method of example 24, further comprising delivering a guidewire through a lumen of the neuromodulation device to the treatment site, wherein delivering neuromodulation energy to the renal nerves comprises delivering energy to the treatment site via at least one energy delivery element disposed on the guidewire.

26. The method of example 24 or example 25, further comprising transforming the distal portion of the neuromodulation device to a spiral shape at the treatment site such that at least one of the energy delivery elements contacts an inner wall of the distal vascular structure at the treatment site before delivering neuromodulation energy.

27. The method of any one of examples 24 to 26, further comprising delivering a guidewire to the treatment site within the distal vascular structure of the renal blood vessel.

28. The method of any one of examples 24 to 27 wherein delivering neuromodulation energy to renal nerves comprises delivering a first energy to energy delivery elements at the first section and a second energy to energy delivery elements at the second section, wherein the first energy is greater than the second energy.

29. The method of any one of examples 24 to 28, further comprising transforming the first section of the distal portion to a first spiral shape and the second section of the distal portion to a second spiral shape, wherein the first section is proximal to the second section, and wherein the first spiral shape has a first outer diameter and the second spiral shape has a second outer diameter less than the first outer diameter.

30. The method of any one of examples 24 to 29, further comprising positioning the second section of the distal portion of the neuromodulation device into a portion of the distal vascular structure having a diameter of less than 3 mm.

### VI. Conclusion

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Certain aspects of the present technology may take the form of computer-executable instructions, including routines executed by a controller or other data processor. In some embodiments, a controller or other data processor is specifically programmed, configured, and/or constructed to perform one or more of these computer-executable instructions. Furthermore, some aspects of the present technology may take the form of data (e.g., non-transitory data) stored or distributed on computer-readable media, including magnetic or optically readable and/or removable computer discs as well as media distributed electronically over networks. Accordingly, data structures and transmissions of data particular to aspects of the present technology are encompassed within the scope of the present technology. The present technology also encompasses methods of both programming computer-readable media to perform particular steps and executing the steps.

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

I/We claim:

1. A neuromodulation device, comprising:
an elongated shaft having a distal portion with a cross-sectional area and a distal end, the cross-sectional area decreasing along a length of the distal portion toward the distal end;
a neuromodulation assembly at the distal portion of the elongated shaft, the neuromodulation assembly comprising a plurality of energy delivery elements longitudinally spaced along the distal portion of the elongated shaft, wherein the plurality of energy delivery elements comprises at least a first electrode and a second electrode distal to the first electrode, and wherein the second electrode has a cross-sectional area less than that of the first electrode; and
a plurality of pairs of lead wires carried by a portion of the elongated shaft, each pair of lead wires coupled to an energy delivery element,
wherein the distal portion of the shaft is sized and shaped for delivery into a distal vascular portion.

2. The neuromodulation device of claim 1 wherein the elongated shaft further comprises a braided circumferential wall, and wherein the lead wires are integrated with the braided circumferential wall.

3. The neuromodulation device of claim 1 wherein the cross-sectional area changes decrementally along the distal portion of the distal portion of the elongated shaft, and wherein the decrement relates to a combined cross-sectional area of a pair of the plurality of lead wires.

4. The neuromodulation device of claim 1 wherein the distal portion of the elongated shaft comprises a greater quantity of lead wires at the first electrode than at the second electrode.

5. A neuromodulation device for delivering neuromodulation therapy in a vessel of a human patient, the neuromodulation device comprising:
an elongated shaft having a distal portion with a first section and a second section distal to the first section, the first section having a first outer cross-sectional dimension and the second section having a second outer cross-sectional dimension less than the second outer cross-sectional dimension;
a neuromodulation assembly at the distal portion of the elongated shaft, the neuromodulation assembly comprising—
a first energy delivery element at the first section of the elongated shaft; and
a second energy delivery element at the second section of the elongated shaft.

6. The neuromodulation device of claim 5, further comprising:
a first pair of lead wires electrically coupled to the first energy delivery element; and
a second pair of lead wires electrically coupled to the second energy delivery element.

7. The neuromodulation device of claim 5 wherein:
the first energy delivery element is a first electrode having a first outer cross-sectional dimension; and
the second energy delivery element is a second electrode having a second outer cross-sectional dimension less than the first outer cross-sectional dimension of the first electrode.

8. The neuromodulation device of claim 5 wherein the elongated shaft comprises a braid.

9. The neuromodulation device of claim 8 wherein a first pair and a second pair of lead wires are incorporated into the braid.

10. The neuromodulation device of claim 5 wherein the distal portion of the elongated shaft includes a shaped portion having a helical shape when the distal portion is in an expanded state.

11. The neuromodulation device of claim 5 wherein the first section is a first shaped portion and the second section is a second shaped portion, and wherein the first and second shaped portions are helical when the distal portion is in an expanded state.

12. The neuromodulation device of claim 11 wherein the first and second shaped sections are separated by a linear portion.

13. The neuromodulation device of claim 5 wherein the elongated shaft comprises a guidewire lumen terminating in an opening at the distal portion of the elongated shaft, and wherein the neuromodulation device further comprises:
a guidewire configured to extend through the guidewire lumen and through the opening; and
a guidewire energy delivery element positioned at a distal section of the guidewire.

14. The neuromodulation device of claim 13 wherein the guidewire further comprises at least one pair of lead wires electrically coupled to the guidewire energy delivery element.

15. The neuromodulation device of claim 13 wherein the distal section of the guidewire defines the second section of the distal portion of the elongated shaft.

16. The neuromodulation device of claim 13 wherein the guidewire has an outer cross-sectional dimension of 0.36 mm (0.014 inch).

17. The neuromodulation device of claim 5, further comprising an atraumatic tip at the distal portion of the elongated shaft.

18. The neuromodulation device of claim 5 wherein the distal portion of the elongated shaft has a distal end, and wherein the neuromodulation assembly further comprises a third energy delivery element at the distal end.

19. The neuromodulation device of claim 5 wherein the elongated shaft further comprises a pre-formed nitinol core configured to impart a shape to the distal portion of the elongated shaft when the distal portion is in an expanded state.

20. The neuromodulation device of claim 5 wherein the elongated shaft, the first energy delivery element, the second energy delivery element, the first pair of lead wires, and the second pair of lead wires are integral components of a single catheter.

21. The neuromodulation device of claim 5 wherein the distal vascular portion is a distal portion of renal vasculature of a human, and wherein the second energy delivery element is configured to deliver energy to ablate renal nerves along the distal vascular portion.

22. The neuromodulation device of claim 5 wherein the first outer cross-sectional dimension is a first outer diameter, and wherein the second outer cross-sectional dimension is a second outer diameter.

23. The neuromodulation device of claim 5 wherein:
the first and second sections of the distal portion of the elongated shaft are two of a plurality of sections of the distal portion of the elongated shaft;
the first and second energy delivery elements are two of a plurality of energy delivery elements positioned at corresponding sections of the distal portion of the elongated shaft; and
each section and each energy delivery element have decrementally changing outer cross-sectional dimensions as the sections are positioned closer to a distal end of the elongated shaft.

24. A method of administering neuromodulation therapy to a patient, the method comprising:
delivering a distal portion of a neuromodulation device to a treatment site within a proximal vascular portion and a distal vascular portion of a renal vascular structure of a human, the distal portion of the neuromodulation device having a plurality of energy delivery elements spaced along a length of the distal portion, wherein the distal portion of the neuromodulation device has a first outer cross-sectional dimension at a first section of the distal portion and a second outer cross-sectional dimension at a second section of the distal portion, wherein the second section is distal to the first section, and wherein the first outer cross-sectional dimension is greater than the second outer cross-sectional dimension; and
delivering neuromodulation energy to renal nerves within the proximal vascular portion and/or the distal vascular portion via at least one energy delivery element of the neuromodulation device.

25. The method of claim 24, further comprising delivering a guidewire through a lumen of the neuromodulation device to the treatment site, wherein delivering neuromodulation energy to the renal nerves comprises delivering energy to the treatment site via at least one energy delivery element disposed on the guidewire.

26. The method of claim 24, further comprising transforming the distal portion of the neuromodulation device to a spiral shape at the treatment site such that at least one of the energy delivery elements contacts an inner wall of the distal vascular structure at the treatment site before delivering neuromodulation energy.

27. The method of claim 24, further comprising delivering a guidewire to the treatment site within the distal vascular portion of a renal blood vessel.

28. The method of claim 24 wherein delivering neuromodulation energy to renal nerves comprises delivering a first energy to energy delivery elements at the first section and a second energy to energy delivery elements at the second section, wherein the first energy is greater than the second energy.

29. The method of claim 24, further comprising transforming the first section of the distal portion to a first spiral shape and the second section of the distal portion to a second spiral shape, wherein the first section is proximal to the second section, and wherein the first spiral shape has a first outer diameter and the second spiral shape has a second outer diameter greater than the first outer diameter.

30. The method of claim 24, further comprising positioning the second section of the distal portion of the neuromodulation device into a portion of the auxiliary branch of the renal blood vessel having a diameter of less than 3 mm.

31. The method of claim 24 wherein delivering the neuromodulation device to the treatment site comprises positioning the portion of the neuromodulation device having the second cross-sectional dimension in the distal vascular portion while the portion of the neuromodulation device having the first cross-sectional dimension is in the proximal vascular portion.

32. The method of claim 24 wherein the proximal vascular portion is along a main renal artery and the distal vascular portion is along an auxiliary branch of the renal artery, and wherein delivering the neuromodulation device to the treatment site comprises positioning the portion of the neuromodulation device having the second cross-sectional dimension in the auxiliary branch while the portion of the neuromodulation device having the first cross-sectional dimension is in the main renal artery.
